(19) United States
(12) Patent Application Publication
(10) Pub. No.: US 2010/0120628 A1
(43) Pub. Date: May 13, 2010

(54) GENEMAP OF THE HUMAN GENES ASSOCIATED WITH ADHD

(75) Inventors: Abdelmajid Belouchi, Beaconsfield (CA); Vanessa Bruat, Montreal (CA); Pascal Croteau, Laval (CA); Daniel Dubois, Laval (CA); Randall David Little, Dorothee (CA); Bruno Paquin, Chateauguay (CA); John Verner Raelson, Hudson Heights (CA); Jonathan Segal, Efrat (IL); Paul Van Eerdewegh, Carlisle, MA (US); Sandie Briand, Montreal (CA); Sem Kebache, Mont-Royal (CA); Tim Keith, Bedford, MA (US)

Correspondence Address: DOWELL & DOWELL P.C., 103 Oronoco St., Suite 220, Alexandria, VA, 22314, US

(73) Assignee: GENIZON BIOSCIENCES INC., Ville St. Laurent, QC (CA)

(21) Appl. No.: 12/449,396

(22) PCT Filed: Feb. 6, 2008
(86) PCT No.: PCT/US08/01528

§ 371 Date: Nov. 12, 2009

Related U.S. Application Data
(60) Provisional application No. 60/899,619, filed on Feb. 6, 2007.

(51) Int. Cl. C40B 30/04; C12Q 1/68

(52) U.S. Cl. 506/9; 435/6

(57) ABSTRACT

The present invention relates to the selection of a set of polymorphism markers for use in genome wide association studies based on linkage disequilibrium mapping. In particular, the invention relates to the fields of pharmacogenomics, diagnostics, patient therapy and the use of genetic haplotype information to predict an individual's susceptibility to ADHD disease and/or their response to a particular drug or drugs.

### PRIORITY

This application is entitled to priority to U.S. Provisional Application No. 60/899,619, filed Feb. 6, 2007, which is hereby incorporated by reference in its entirety.

### FIELD OF THE INVENTION

The invention relates to the field of genomics and genetics, including genome analysis and the study of DNA variations. In particular, the invention relates to the fields of pharmacogenomics, diagnostics, patient therapy and the use of genetic haplotype information to predict an individual's susceptibility to ADHD disease and/or their response to a particular drug or drugs, so that drugs tailored to genetic differences of population groups may be developed and/or administered to the appropriate population.

The invention also relates to a GeneMap for ADHD disease, which links variations in DNA (including both genic and non-genic regions) to an individual's susceptibility to ADHD disease and/or response to a particular drug or drugs. The invention further relates to the genes disclosed in the GeneMap (see Tables 2-4), which is related to methods and reagents for detection of an individual's increased or decreased risk for ADHD disease and related sub-phenotypes, by identifying at least one polymorphism in one or a combination of the genes from the GeneMap. Also related are the candidate regions identified in Table 1, which are associated with ADHD disease. In addition, the invention further relates to nucleotide sequences of those genes including genomic DNA sequences, DNA sequences, single nucleotide polymorphisms (SNPs), other types of polymorphisms (insertions, deletions, microsatellites), alleles and haplotypes (see Sequence Listing and Tables 5-37).

The invention further relates to isolated nucleic acids comprising these nucleotide sequences and isolated polypeptides or peptides encoded thereby. Also related are expression vectors and host cells comprising the disclosed nucleic acids or fragments thereof, as well as antibodies that bind to the encoded polypeptides or peptides.

The present invention further relates to ligands that modulate the activity of the disclosed genes or gene products. In addition, the invention relates to diagnostics and therapeutics for ADHD disease, utilizing the disclosed nucleic acids, polymorphisms, chromosomal regions, gene maps, polypeptides or peptides, antibodies and/or ligands and small molecules that activate or repress relevant signaling events.

### BACKGROUND OF THE INVENTION

Attention-deficit/hyperactivity disorder (ADHD) is the most common heritable and familial neuropsychiatric disorder that affects 3-5% worldwide and 2-12% in Canada of school-aged children, with a higher incidence in boys with a ratio between 3:1 to 9:1. Its name reflects the range of possible clinical presentations, which include hyperactivity, forgetfulness, mood shifts, poor impulse control, and distractibility. ADHD is divided into three subtypes; the predominantly inattentive subtype, the predominantly hyperactive-impulsive subtype and the combined subtype. Eight percent of diagnosed children display a mix of all three symptoms. However, the inattentive subtype is the most prevalent. Subjects with ADHD have higher frequency of school failures due to learning disorders, unsociability, greater risk of substance abuse and oppositional defiant behavior. It is believed that between 30 to 70% of children diagnosed with ADHD retain the disorder as adults.

In neurological pathology, ADHD is currently believed to be a chronic syndrome for which no medical cure is available. Moreover, it is also considered a genetically complex disorder since it does not follow classical Mendelian segregation. Although the precise neural and pathophysiological mechanisms remain unknown, neuro-imaging, animal models and pharmacological studies suggest the involvement of the dopaminergic neurotransmitter pathways. The genes encoding the dopamine receptors and transporters such as the dopamine transporter gene (DAT1), the dopamine receptor 4 and 5 gene (DRD4, DRD5), have been the most attractive candidate genes for ADHD, as determined by the candidate gene approach. Recent studies have also implicated brain catecholamine systems in ADHD pathophysiological and pharmacological interventions, especially their relevance in the prefrontal cortex (PFC), the brain area that guides executive functions mainly behavior, thought, and working memory. Lesions to the PFC or inadequate catecholamine transmission produce symptoms similar to ADHD. Methylphenidate, amphetamine and atomoxetine, drugs used for treating ADHD, attenuate catecholamine transporter function, thereby enhancing dopamine and norepinephrine transmission in PFC. These drugs are considered powerful stimulants with a potential for diversion and abuse, therefore, there is controversy surrounding prescribing these drugs for children and adolescents.

To date, three independent genome scans of ADHD have been performed, which examined allele sharing in affected sibling pairs with an average marker spacing of 10 cm, while a fourth genome scan was recently published which examined allele sharing in extended multigenerational pedigrees. Two of the studies showed the linkage of three chromosomal regions (i.e., 5q13, 11q22-25 and 17p11), which contain several candidate genes including DRD4 and DAT1.

Current treatments for ADHD disease are primarily aimed at reducing symptoms and do not address the root cause of the disease. Despite a preponderance of evidence showing inheritance of a risk for ADHD disease through epidemiological studies and genome wide linkage analyses, the genes affecting ADHD disease have yet to be discovered (Hugot J P, and Thomas G., 1998). There is a need in the art for identifying specific genes related to ADHD disease to enable the development of therapeutics that address the causes of the disease rather than relieving its symptoms. The failure in past studies to identify causative genes in complex diseases, such as ADHD disease, has been due to the lack of appropriate methods to detect a sufficient number of variations in genomic DNA samples (markers), the insufficient quantity of necessary markers available, and the number of needed individuals to enable such a study. The present invention addresses these issues.

The present invention relates specifically to a set of ADHD disease-causing genes (GeneMap) and targets which present attractive points of therapeutic intervention.

In view of the foregoing, identifying susceptibility genes associated with ADHD disease and their respective biochemical pathways will facilitate the identification of diagnostic markers as well as novel targets for improved therapeutics. It will also improve the quality of life for those afflicted by this disease and will reduce the economic costs of these afflictions at the individual and societal level. The identification of those genetic markers would provide the basis for novel genetic tests and eliminate or reduce the therapeutic methods currently used. The identification of those genetic markers will also provide the development of effective therapeutic intervention for the battery of laboratory, psychological and clinical evaluations typically required to diagnose ADHD disease. The present invention satisfies this need.

LENGTHY TABLES
The patent application contains a lengthy table section. A copy of the table is available in electronic form from the USPTO web site (http://seqdata.uspto.gov/?pageRequest=docDetail&DocID=US20100120628A1). An electronic copy of the table will also be available from the USPTO upon request and payment of the fee set forth in 37 CFR 1.19(b)(3).

### DESCRIPTION OF THE FILES CONTAINED ON THE CD-R

The contents of the submission on compact discs submitted herewith are incorporated herein by reference in their entirety: A compact disc copy of the Sequence Listing (COPY 1) (filename: GENI 023 01WO SeqList.txt, date recorded: Feb. 6, 2008, file size: 41,523 kilobytes); a duplicate compact disc copy of the Sequence Listing (COPY 2) (filename: GENI 023 01WO SeqList.txt, date recorded: Feb. 6, 2008, file size: 41,523 kilobytes); a duplicate compact disc copy of the Sequence Listing (COPY 3) (filename: GENI 023 01WO SeqList.txt, date recorded: Feb. 6, 2008, file size: 41,523 kilobytes); a computer readable format copy of the Sequence Listing (CRF COPY) (filename: GENI 023 01WO SeqList.txt, date recorded: Feb. 6, 2008; file size: 41,523 kilobytes).

Three compact disc copies (COPY 1, COPY 2 and COPY3) of Tables 1-38 are herewith submitted and are incorporated herein by reference in their entirety. Each compact disc contains a copy of the following files:

filename: Table1.txt, date recorded: Feb. 6, 2008, file size: 27 kilobytes;

filename: Table2.txt, date recorded: Feb. 6, 2008, file size: 118 kilobytes;

filename: Table3.txt, date recorded: Feb. 6, 2008, file size: 278 kilobytes;

filename: Table4.txt, date recorded: Feb. 6, 2008, file size: 2 kilobytes;

filename: Table5.1.txt, date recorded: Feb. 6, 2008, file size: 318 kilobytes;

filename: Table5.2.txt, date recorded: Feb. 6, 2008, file size: 673 kilobytes;

filename: Table6.1.txt, date recorded: Feb. 6, 2008, file size: 11 kilobytes;

filename: Table6.2.txt, date recorded: Feb. 6, 2008, file size: 30 kilobytes;

filename: Table7.1.txt, date recorded: Feb. 6, 2008, file size: 15 kilobytes;

filename: Table7.2.txt, date recorded: Feb. 6, 2008, file size: 11 kilobytes;

filename: Table8.1.txt, date recorded: Feb. 6, 2008, file size: 7 kilobytes;

filename: Table8.2.txt, date recorded: Feb. 6, 2008, file size: 5 kilobytes;

filename: Table9.1, date recorded: Feb. 6, 2008, file size: 4 kilobytes;

filename: Table9.2, date recorded: Feb. 6, 2008, file size: 19 kilobytes;

filename: Table10.1, date recorded: Feb. 6, 2008, file size: 7 kilobytes;

filename: Table10.2, date recorded: Feb. 6, 2008, file size: 5 kilobytes;

filename: Table11.1, date recorded: Feb. 6, 2008, file size: 4 kilobytes;

filename: Table11.2, date recorded: Feb. 6, 2008, file size: 7 kilobytes;

filename: Table12.1, date recorded: Feb. 6, 2008, file size: 17 kilobytes;

filename: Table12.2, date recorded: Feb. 6, 2008, file size: 43 kilobytes;

filename: Table13.1, date recorded: Feb. 6, 2008, file size: 9 kilobytes;

filename: Table13.2, date recorded: Feb. 6, 2008, file size: 22 kilobytes;

filename: Table14.1, date recorded: Feb. 6, 2008, file size: 12 kilobytes;

filename: Table14.2, date recorded: Feb. 6, 2008, file size: 4 kilobytes;

filename: Table15.1, date recorded: Feb. 6, 2008, file size: 45 kilobytes;

filename: Table15.2, date recorded: Feb. 6, 2008, file size: 80 kilobytes;

filename: Table16.1, date recorded: Feb. 6, 2008, file size: 35 kilobytes;

filename: Table16.2, date recorded: Feb. 6, 2008, file size: 75 kilobytes;

filename: Table17.1, date recorded: Feb. 6, 2008, file size: 6 kilobytes;

filename: Table17.2, date recorded: Feb. 6, 2008, file size: 32 kilobytes;

filename: Table18.1, date recorded: Feb. 6, 2008, file size: 28 kilobytes;

filename: Table18.2, date recorded: Feb. 6, 2008, file size: 76 kilobytes;

filename: Table19.1, date recorded: Feb. 6, 2008, file size: 9 kilobytes;

filename: Table19.2, date recorded: Feb. 6, 2008, file size: 22 kilobytes;

filename: Table20.1, date recorded: Feb. 6, 2008, file size: 46 kilobytes;

filename: Table20.2, date recorded: Feb. 6, 2008, file size: 40 kilobytes;

filename: Table20.3, date recorded: Feb. 6, 2008, file size: 104 kilobytes;

filename: Table21.1, date recorded: Feb. 6, 2008, file size: 59 kilobytes;

filename: Table21.2, date recorded: Feb. 6, 2008, file size: 45 kilobytes;

filename: Table21.3, date recorded: Feb. 6, 2008, file size: 218 kilobytes;

filename: Table22.1, date recorded: Feb. 6, 2008, file size: 103 kilobytes;

filename: Table22.2, date recorded: Feb. 6, 2008, file size: 95 kilobytes;

filename: Table22.3, date recorded: Feb. 6, 2008, file size: 334 kilobytes;

filename: Table23.1, date recorded: Feb. 6, 2008, file size: 52 kilobytes;

filename: Table23.2, date recorded: Feb. 6, 2008, file size: 40 kilobytes;

filename: Table23.3, date recorded: Feb. 6, 2008, file size: 140 kilobytes;

filename: Table24.1, date recorded: Feb. 6, 2008, file size: 20 kilobytes;

filename: Table24.2, date recorded: Feb. 6, 2008, file size: 18 kilobytes;

filename: Table24.3, date recorded: Feb. 6, 2008, file size: 46 kilobytes;

filename: Table25.1, date recorded: Feb. 6, 2008, file size: 23 kilobytes;

filename: Table25.2, date recorded: Feb. 6, 2008, file size: 20 kilobytes;

filename: Table25.3, date recorded: Feb. 6, 2008, file size: 49 kilobytes;

filename: Table26.1, date recorded: Feb. 6, 2008, file size: 10 kilobytes;

filename: Table26.2, date recorded: Feb. 6, 2008, file size: 8 kilobytes;

filename: Table26.3, date recorded: Feb. 6, 2008, file size: 19 kilobytes;

filename: Table27.1, date recorded: Feb. 6, 2008, file size: 153 kilobytes;

filename: Table27.2, date recorded: Feb. 6, 2008, file size: 122 kilobytes;

filename: Table27.3, date recorded: Feb. 6, 2008, file size: 304 kilobytes;

filename: Table28.1, date recorded: Feb. 6, 2008, file size: 65 kilobytes;

filename: Table28.2, date recorded: Feb. 6, 2008, file size: 50 kilobytes;

filename: Table28.3, date recorded: Feb. 6, 2008, file size: 474 kilobytes;

filename: Table29.1, date recorded: Feb. 6, 2008, file size: 2 kilobytes;

filename: Table29.2, date recorded: Feb. 6, 2008, file size: 2 kilobytes;

filename: Table30.1, date recorded: Feb. 6, 2008, file size: 13 kilobytes;

filename: Table30.2, date recorded: Feb. 6, 2008, file size: 12 kilobytes;

filename: Table30.3, date recorded: Feb. 6, 2008, file size: 37 kilobytes;

filename: Table31.1, date recorded: Feb. 6, 2008, file size: 26 kilobytes;

filename: Table31.2, date recorded: Feb. 6, 2008, file size: 70 kilobytes;

filename: Table32.1, date recorded: Feb. 6, 2008, file size: 55 kilobytes;

filename: Table32.2, date recorded: Feb. 6, 2008, file size: 39 kilobytes;

filename: Table32.3, date recorded: Feb. 6, 2008, file size: 118 kilobytes;

filename: Table33.1, date recorded: Feb. 6, 2008, file size: 143 kilobytes;

filename: Table33.2, date recorded: Feb. 6, 2008, file size: 119 kilobytes;

filename: Table33.3, date recorded: Feb. 6, 2008, file size: 195 kilobytes;

filename: Table34.1, date recorded: Feb. 6, 2008, file size: 96 kilobytes;

filename: Table34.2, date recorded: Feb. 6, 2008, file size: 68 kilobytes;

filename: Table34.3, date recorded: Feb. 6, 2008, file size: 131 kilobytes;

filename: Table35.1, date recorded: Feb. 6, 2008, file size: 29 kilobytes;

filename: Table35.2, date recorded: Feb. 6, 2008, file size: 12 kilobytes;

filename: Table36.1, date recorded: Feb. 6, 2008, file size: 69 kilobytes;

filename: Table36.2, date recorded: Feb. 6, 2008, file size: 52 kilobytes;

filename: Table36.3, date recorded: Feb. 6, 2008, file size: 160 kilobytes;

filename: Table37.1, date recorded: Feb. 6, 2008, file size: 6 kilobytes;

filename: Table37.2, date recorded: Feb. 6, 2008, file size: 4 kilobytes; and filename: Table38, date recorded: Feb. 6, 2008, file size: 16 kilobytes.

### Table Description

Table 1. List of ADHD candidate regions identified from the Genome Wide Scan association analyses. The first column denotes the region identifier. The second and third columns correspond to the chromosome and cytogenetic band, respectively. The fourth and fifth columns correspond to the chromosomal start and end coordinates of the NCBI genome assembly derived from build 36.

Table 2. List of candidate genes from the regions identified from the genome wide association analysis. The first column corresponds to the region identifier provided in Table 1. The second and third columns correspond to the chromosome and cytogenetic band, respectively. The fourth and fifth columns corresponds to the chromosomal start coordinates of the NCBI genome assembly derived from build 36 (B36) and the end coordinates (the start and end position relate to the +orientation of the NCBI assembly and don't necessarily correspond to the orientation of the gene). The sixth and seventh columns correspond to the official gene symbol and gene name, respectively, and were obtained from the NCBI Entrez Gene database. The eighth column corresponds to the NCBI Entrez Gene Identifier (GeneID). The ninth and tenth columns correspond to the Sequence IDs from nucleotide (cDNA) and protein entries in the Sequence Listing.

Table 3. List of candidate genes based on EST clustering from the regions identified from the various genome wide analyses. The first column corresponds to the region identifier provided in Table 1. The second column corresponds to the chromosome number. The third and fourth columns correspond to the chromosomal start and end coordinates of the NCBI genome assemblies derived from build 36 (B36). The fifth column corresponds to the ECGene Identifier, corresponding to the ECGene track of UCSC. These ECGene entries were determined by their overlap with the regions from Table 1, based on the start and end coordinates of both Region and ECGene identifiers. The sixth and seventh columns correspond to the Sequence IDs from nucleotide and protein entries in the Sequence Listing.

Table 4. List of micro RNA (miRNA) from the regions identified from the genome wide association analyses derived from build 36 (B36). To identify the miRNA from B36, these miRNA entries were determined by their overlap with the regions from Table 1, based on the start and end coordinates of both Region and miRNA identifiers. The first column corresponds to the region identifier provided in Table 1. The second column corresponds to the chromosome number. The third and fourth columns correspond to the chromosomal start and end coordinates of the NCBI genome assembly derived from build 36 (the start and end position relate to the +orientation of the NCBI assembly and do not necessarily correspond to the orientation of the miRNA). The fifth and sixth columns correspond to the miRNA accession and miRNA id, respectively, and were obtained from the miRBase database. The seventh column corresponds to the NCBI Entrez Gene Identifier (GeneID). The eighth column corresponds to the Sequence ID from nucleotide (RNA) in the Sequence Listing.

Table 5.1. Genome wide association study results in the Quebec Founder Population (QFP). SNP markers found to be associated with ADHD from the analysis of genome wide scan (GWS) data: full cohort. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 5.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 5.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 6.1. Genome wide association study results in the Quebec Founder Population (QFP). SNP markers found to be associated with ADHD from the analysis of genome wide scan (GWS) data: HasGRID1-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 6.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 6.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 7.1. Genome wide association study results in the Quebec Founder Population (QFP). SNP markers found to be associated with ADHD from the analysis of genome wide scan (GWS) data: HasTAF4-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 7.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 7.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 8.1. Genome wide association study results in the Quebec Founder Population (QFP). SNP markers found to be associated with ADHD from the analysis of genome wide scan (GWS) data: HasSLC6A14-1_cp2. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 8.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 8.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 9.1. Genome wide association study results in the Quebec Founder Population (QFP). SNP markers found to be associated with ADHD from the analysis of genome wide scan (GWS) data: HasSLC6A14-1a_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 9.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 9.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 10.1. Genome wide association study results in the Quebec Founder Population (QFP). SNP markers found to be associated with ADHD from the analysis of genome wide scan (GWS) data: NotLOC643182-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 10.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 10.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 11.1. Genome wide association study results in the Quebec Founder Population (QFP). SNP markers found to be associated with ADHD from the analysis of genome wide scan (GWS) data: NotKCNAB1-1-cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 11.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 11.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 12.1. Genome wide association study results in the Quebec Founder Population (QFP). SNP markers found to be associated with ADHD from the analysis of genome wide scan (GWS) data: NotLOC643182-1_cp. Columns include: Region ID; Chromosome; Build 36 location in base pairs (bp); rs#, dbSNP data base (NCBI) reference number; Sequence. ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 12.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 12.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 13.1. Genome wide association study results in the Quebec Founder Population (QFP). SNP markers found to be associated with ADHD from the analysis of genome wide scan (GWS) data: NotTAF4-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 13.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 13.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 14.1. Genome wide association study results in the Quebec Founder Population (QFP). SNP markers found to be associated with ADHD from the analysis of genome wide scan (GWS) data: NotTAF4-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 14.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 14.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 15.1. Genome wide association study results in the Quebec Founder Population (QFP). SNP markers found to be associated with ADHD from the analysis of genome wide scan (GWS) data: NotSLC6A14-1_cp2. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 15.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 15.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 16.1. Genome wide association study results in the Quebec Founder Population (QFP). SNP markers found to be associated with ADHD from the analysis of genome wide scan (GWS) data: AFFECTED FEMALE. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 16.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 16.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 17.1. Genome wide association study results in the Quebec Founder Population (QFP). SNP markers found to be associated with ADHD from the analysis of genome wide scan (GWS) data: NotSLC6414-1_cr2. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 17.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 17.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 18.1. Genome wide association study results in the Quebec Founder Population (QFP). SNP markers found to be associated with ADHD from the analysis of genome wide scan (GWS) data: NotSLC6A14-1a_cp1. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 18.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 18.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 19.1. Genome wide association study results in the Quebec Founder Population (QFP). SNP markers found to be associated with ADHD from the analysis of genome wide scan (GWS) data: NotSLC6A14-1A_cr1. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 19.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 19.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 20.1. ALL the Genome wide association study results in the Quebec Founder Population (QFP) (including SNPs out of CR from Table 1). SNP markers found to be associated with ADHD from the analysis of genome wide scan (GWS) data: HASODZ3-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 20.2. Genome wide association study results in the Quebec Founder Population (QFP). SNP markers found to be associated with ADHD from the analysis of genome wide scan (GWS) data: HASODZ3-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 20.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 20.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 21.1. ALL the Genome wide association study results in the Quebec Founder Population (QFP) (including SNPs out of CR from Table 1). SNP markers found to be associated with ADHD from the analysis of genome wide scan (GWS) data: HASODZ3-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 21.2. ALL the Genome wide association study results in the Quebec Founder Population (QFP) (including SNPs out of CR from Table 1). SNP markers found to be associated with ADHD from the analysis of genome wide scan (GWS) data: HAS-ODZ3-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 21.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 21.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 22.1. ALL the Genome wide association study results in the Quebec Founder Population (QFP) (including SNPs out of CR from Table 1). SNP markers found to be associated with ADHD from the analysis of genome wide scan (GWS) data: HAS-ODZ3-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 22.2. Genome wide association study results in the Quebec Founder Population (QFP). SNP markers found to be associated with ADHD from the analysis of genome wide scan (GWS) data: HAS-ODZ3-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 22.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 22.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 23.1. ALL the Genome wide association study results in the Quebec Founder Population (QFP) (including SNPs out of CR from Table 1). SNP markers found to be associated with ADHD from the analysis of genome wide scan (GWS) data: HAS-ODZ3-2_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 23.2. Genome wide association study results in the Quebec Founder Population (QFP). SNP markers found to be associated with ADHD from the analysis of genome wide scan (GWS) data: HAS-ODZ3-2_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 23.3. List of significantly associated haplotypes based on the ADHD results using the Quebec Founder Population (QFP). Individual haplotypes with associated relative risks are presented in each row of the table; these values were extracted from the associated marker haplotype window with the most significant p value for each SNP in Table 23.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 24.1. ALL the Genome wide association study results in the Quebec Founder Population (QFP) (including SNPs out of CR from Table 1). SNP markers found to be associated with ADHD from the analysis of genome wide scan (GWS) data: HAS-ODZ3-2_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 24.2. Genome wide association study results in the Quebec Founder Population (QFP). SNP markers found to be associated with ADHD from the analysis of genome wide scan (GWS) data: HAS-ODZ3-2_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 24.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 24.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 25.1. ALL the Genome wide association study results in the Quebec Founder Population (QFP) (including SNPs out of CR from Table 1). SNP markers found to be associated with ADHD from the analysis of genome wide scan (GWS) data: NOT-ODZ3-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 25.2. Genome wide association study results in the Quebec Founder Population (QFP). SNP markers found to be associated with ADHD from the analysis of genome wide scan (GWS) data: NOT-ODZ3-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 25.3. List of significantly associated haplotypes based on the ADHD results using the Quebec Founder Population (QFP). Individual haplotypes with associated relative risks are presented in each row of the table; these values were extracted from the associated marker haplotype window with the most significant p value for each SNP in Table 25.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 26.1. ALL the Genome wide association study results in the Quebec Founder Population (QFP) (including SNPs out of CR from Table 1). SNP markers found to be associated with ADHD from the analysis of genome wide scan (GWS) data: NOT-ODZ3-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 26.2. Genome wide association study results in the Quebec Founder Population (QFP). SNP markers found to be associated with ADHD from the analysis of genome wide scan (GWS) data: NOT-ODZ3-1_cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 26.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 26.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 27.1. ALL the Genome wide association study results in the Quebec Founder Population (QFP) (including SNPs out of CR from Table 1). SNP markers found to be associated with ADHD from the analysis of genome wide scan (GWS) data: Affected male. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 27.2. Genome wide association study results in the Quebec Founder Population (QFP). SNP markers found to be associated with ADHD from the analysis of genome wide scan (GWS) data: Affected male. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 27.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 27.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 28.1. ALL the Genome wide association study results in the Quebec Founder Population (QFP) (including SNPs out of CR from Table 1). SNP markers found to be associated with ADHD from the analysis of genome wide scan (GWS) data: Not-ODZ3-1-cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 28.2. Genome wide association study results in the Quebec Founder Population (QFP). SNP markers found to be associated with ADHD from the analysis of genome wide scan (GWS) data: Not-ODZ3-1-cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 28.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 28.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 29.1. ALL the Genome wide association study results in the Quebec Founder Population (QFP) (including SNPs out of CR from Table 1). SNP markers found to be associated with ADHD from the analysis of genome wide scan (GWS) data: Not-ODZ3-2-cp. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 29.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 29.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 30.1. ALL the Genome wide association study results in the Quebec Founder Population (QFP) (including SNPs out of CR from Table 1). SNP markers found to be associated with ADHD from the analysis of genome wide scan (GWS) data: Not-ODZ3-2-cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 30.2. Genome wide association study results in the Quebec Founder Population (QFP). SNP markers found to be associated with ADHD from the analysis of genome wide scan (GWS) data: Not-ODZ3-2-cr. Columns include:

Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 30.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 31.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 31.1. Genome wide association study results in the Quebec Founder Population (QFP). SNP markers found to be associated with ADHD from the analysis of genome wide scan (GWS) data: Not-GRID1-1-cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 31.2. List of significantly associated haplotypes based on the ADHD results using the Quebec Founder Population (QFP). Individual haplotypes with associated relative risks are presented in each row of the table; these values were extracted from the associated marker haplotype window with the most significant p value for each SNP in Table 31.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 32.1. All the Genome wide association study results in the Quebec Founder Population (QFP) including markers outise of the CR from table 1. SNP markers found to be associated with ADHD from the analysis of genome wide scan (GWS) data: Hascombinedsub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 32.2. Genome wide association study results in the Quebec Founder Population (QFP). SNP markers found to be associated with ADHD from the analysis of genome wide scan (GWS) data: Hascombinedsub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 32.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 32.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 33.1. All the Genome wide association study results in the Quebec Founder Population (QFP) including markers outise of the CR from table 1. SNP markers found to be associated with ADHD from the analysis of genome wide scan (GWS) data: Hasinattentivesub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 33.2. Genome wide association study results in the Quebec Founder Population (QFP). SNP markers found to be associated with ADHD from the analysis of genome wide scan (GWS) data: Hasinattentivesub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 33.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 33.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 34.1. All the Genome wide association study results in the Quebec Founder Population (QFP) including markers outise of the CR from table 1. SNP markers found to be associated with ADHD from the analysis of genome wide scan (GWS) data: Notcombinedsub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 34.2. Genome wide association study results in the Quebec Founder Population (QFP). SNP markers found to be associated with ADHD from the analysis of genome wide scan (GWS) data: Notcombinedsub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 34.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 34.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 35.1. Genome wide association study results in the Quebec Founder Population (QFP). SNP markers found to be associated with ADHD from the analysis of genome wide scan (GWS) data: Nothyperactivesub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 35.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 35.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 36.1. All the Genome wide association study results in the Quebec Founder Population (QFP) including markers outside of CR in Table 1. SNP markers found to be associated with ADHD from the analysis of genome wide scan (GWS) data: Notinattentivesub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 36.2. Genome wide association study results in the Quebec Founder Population (QFP). SNP markers found to be associated with ADHD from the analysis of genome wide scan (GWS) data: Notinattentivesub-type. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 36.3. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 36.2. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 37.1. Genome wide association study results in the Quebec Founder Population (QFP). SNP markers found to be associated with ADHD from the analysis of genome wide scan (GWS) data: HAS-LOC643182-1_cr. Columns include: Region ID; Chromosome; Build 36 location in base pairs (bp); rs#, dbSNP data base (NCBI) reference number; Sequence ID, unique numerical identifier for this patent application; Sequence, 21 by of sequence covering 10 base pair of unique sequence flanking either side of central polymorphic SNP; −log 10 P values for GWS, −log 10 of the P value for statistical significance from the GWS for single SNP markers (both T test and Permutation test p-values are displayed; see Example section) and for the most highly associated multi-marker haplotypes centered at the reference marker and defined by the sliding windows of specified sizes.

Table 37.2. List of significantly associated haplotypes based on the ADHD GWS results using the Quebec Founder Population (QFP). Individual haplotypes with associated relative risks are presented in each row of the table; these values were extracted from the associated marker haplotype window with the most significant p value for each SNP in Table 37.1. The first column lists the region ID as presented in Table 1. The Haplotype column lists the specific nucleotides for the individual SNP alleles contributing to the haplotype reported. The Case and Control columns correspond to the numbers of cases and controls, respectively, containing the haplotype variant noted in the Haplotype column. The Total Case and Total Control columns list the total numbers of cases and controls for which genotype data was available for the haplotype in question. The RR column gives to the relative risk for each particular haplotype. The remainder of the columns lists the SeqIDs for the SNPs contributing to the haplotype and their relative location with respect to the central marker. The Central marker (0) column lists the SeqID for the central marker on which the haplotype is based. Flanking markers are identified by minus (−) or plus (+) signs to indicate the relative location of flanking SNPs.

Table 38. Expression study. Semi-quantitative determination of relative mRNA abundance in various tissues (see Example section for details).

### DEFINITIONS

Throughout the description of the present invention, several terms are used that are specific to the science of this field. For the sake of clarity and to avoid any misunderstanding, these definitions are provided to aid in the understanding of the specification and claims.

Allele: One of a pair, or series, of forms of a gene or non-genic region that occur at a given locus in a chromosome. Alleles are symbolized with the same basic symbol (e.g., B for dominant and b for recessive; B1, B2, Bn for n additive alleles at a locus). In a normal diploid cell there are two alleles of any one gene (one from each parent), which occupy the same relative position (locus) on homologous chromosomes. Within a population there may be more than two alleles of a gene. See multiple alleles. SNPs also have alleles, i.e., the two (or more) nucleotides that characterize the SNP.

Amplification of nucleic acids: refers to methods such as polymerase chain reaction (PCR), ligation amplification (or ligase chain reaction, LCR) and amplification methods based on the use of Q-beta replicase. These methods are well known in the art and are described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the disorder region or in regions that flank a target region therein. Genes from Tables 2-4 generated by amplification may be sequenced directly. Alternatively, the amplified sequence(s) may be cloned prior to sequence analysis.

Antigenic component: is a moiety that binds to its specific antibody with sufficiently high affinity to form a detectable antigen-antibody complex.

Antibodies: refer to polyclonal and/or monoclonal antibodies and fragments thereof, and immunologic binding equivalents thereof, that can bind to proteins and fragments thereof or to nucleic acid sequences from the disorder region, particularly from the disorder gene products or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera are tested for immunoreactivity to the protein or fragment. Monoclonal antibodies may be made by injecting mice with the proteins, or fragments thereof. Monoclonal antibodies can be screened by ELISA and tested for specific immunoreactivity with protein or fragments thereof (Harlow et al. 1988, Antibodies: A Laboratory Manual, Cold Spring Harbor Laboratory, Cold Spring Harbor, N.Y.). These antibodies will be useful in developing assays as well as therapeutics.

Associated allele: refers to an allele at a polymorphic locus that is associated with a particular phenotype of interest, e.g., a predisposition to a disorder or a particular drug response.

cDNA: refers to complementary or copy DNA produced from an RNA template by the action of RNA-dependent DNA polymerase (reverse transcriptase). Thus, a cDNA clone means a duplex DNA sequence complementary to an RNA molecule of interest, included in a cloning vector or PCR amplified. This term includes genes from which the intervening sequences have been removed.

cDNA library: refers to a collection of recombinant DNA molecules containing cDNA inserts that together comprise essentially all of the expressed genes of an organism or tissue. A cDNA library can be prepared by methods known to one skilled in the art (see, e.g., Cowell and Austin, 1997, “DNA Library Protocols,” Methods in Molecular Biology). Generally, RNA is first isolated from the cells of the desired organism, and the RNA is used to prepare cDNA molecules.

Cloning: refers to the use of recombinant DNA techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

Cloning vector: refers to a plasmid or phage DNA or other DNA molecule that is able to replicate in a host cell. The cloning vector is typically characterized by one or more endonuclease recognition sites at which such DNA sequences may be cleaved in a determinable fashion without loss of an essential biological function of the DNA, and which may contain a selectable marker suitable for use in the identification of cells containing the vector.

Coding sequence or a protein-coding sequence: is a polynucleotide sequence capable of being transcribed into mRNA and/or capable of being translated into a polypeptide or peptide. The boundaries of the coding sequence are typically determined by a translation start codon at the 5′-terminus and a translation stop codon at the 3′-terminus.

Complement of a nucleic acid sequence: refers to the antisense sequence that participates in Watson-Crick base-pairing with the original sequence.

Disorder region: refers to the portions of the human chromosomes displayed in Table 1 bounded by the markers from Tables 2-37.

Disorder-associated nucleic acid or polypeptide sequence: refers to a nucleic acid sequence that maps to region of Table 1 or the polypeptides encoded therein (Tables 2-4, nucleic acids, and polypeptides). For nucleic acids, this encompasses sequences that are identical or complementary to the gene sequences from Tables 2-4, as well as sequence-conservative, function-conservative, and non-conservative variants thereof. For polypeptides, this encompasses sequences that are identical to the polypeptide, as well as function-conservative and non-conservative variants thereof. Included are the alleles of naturally-occurring polymorphisms causative of ADHD disease such as, but not limited to, alleles that cause altered expression of genes of Tables 2-4 and alleles that cause altered protein levels or stability (e.g., decreased levels, increased levels, expression in an inappropriate tissue type, increased stability, and decreased stability).

Expression vector: refers to a vehicle or plasmid that is capable of expressing a gene that has been cloned into it, after transformation or integration in a host cell. The cloned gene is usually placed under the control of (i.e., operably linked to) a regulatory sequence.

Function-conservative variants: are those in which a change in one or more nucleotides in a given codon position results in a polypeptide sequence in which a given amino acid residue in the polypeptide has been replaced by a conservative amino acid substitution. Function-conservative variants also include analogs of a given polypeptide and any polypeptides that have the ability to elicit antibodies specific to a designated polypeptide.

Founder population: Also a population isolate, this is a large number of people who have mostly descended, in genetic isolation from other populations, from a much smaller number of people who lived many generations ago.

Gene: Refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions, as well as regulatory regions, and can include 5′ and 3′ ends. A gene sequence is wild-type if such sequence is usually found in individuals unaffected by the disorder or condition of interest. However, environmental factors and other genes can also play an important role in the ultimate determination of the disorder. In the context of complex disorders involving multiple genes (oligogenic disorder), the wild type, or normal sequence can also be associated with a measurable risk or susceptibility, receiving its reference status based on its frequency in the general population.

GeneMaps: are defined as groups of gene(s) that are directly or indirectly involved in at least one phenotype of a disorder (some non-limiting example of GeneMaps comprises varius combinations of genes from Tables 2-4). As such, GeneMaps enable the development of synergistic diagnostic products, creating “theranostics”.

Genotype: Set of alleles at a specified locus or loci.

Haplotype: The allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a disorder or condition of interest, e.g., ADHD disease. In other instances, an allele or haplotype may be associated with a decrease in susceptibility to a disorder or condition of interest, i.e., a protective sequence.

Host: includes prokaryotes and eukaryotes. The term includes an organism or cell that is the recipient of an expression vector (e.g., autonomously replicating or integrating vector).

Hybridizable: nucleic acids are hybridizable to each other when at least one strand of the nucleic acid can anneal to another nucleic acid strand under defined stringency conditions. In some embodiments, hybridization requires that the two nucleic acids contain at least 10 substantially complementary nucleotides; depending on the stringency of hybridization, however, mismatches may be tolerated. The appropriate stringency for hybridizing nucleic acids depends on the length of the nucleic acids and the degree of complementarity, and can be determined in accordance with the methods described herein.

Identity by descent (IBD): Identity among DNA sequences for different individuals that is due to the fact that they have all been inherited from a common ancestor. LD mapping identifies IBD haplotypes as the likely location of disorder genes shared by a group of patients.

Identity: as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, Computer Analysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

Immunogenic component: is a moiety that is capable of eliciting a humoral and/or cellular immune response in a host animal.

Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Isolated polypeptides or peptides: are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80% or 90% pure. Isolated polypeptides and peptides include those obtained by methods described herein, or other established methods, including isolation from natural sources (e.g., cells, tissues, or organs), chemical synthesis, recombinant methods, or combinations of recombinant and chemical methods. Proteins or polypeptides referred to herein as recombinant are proteins or polypeptides produced by the expression of recombinant nucleic acids. A portion as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a protein or polypeptide.

Linkage disequilibrium (LD): the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

LD mapping: population based gene mapping, which locates disorder genes by identifying regions of the genome where haplotypes or marker variation patterns are shared statistically more frequently among disorder patients compared to healthy controls. This method is based upon the assumption that many of the patients will have inherited an allele associated with the disorder from a common ancestor (IBD), and that this allele will be in LD with the disorder gene.

Locus: a specific position along a chromosome or DNA sequence. Depending upon context, a locus could be a gene, a marker, a chromosomal band or a specific sequence of one or more nucleotides.

Minor allele frequency (MAF): the population frequency of one of the alleles for a given polymorphism, which is equal or less than 50%. The sum of the MAF and the Major allele frequency equals one.

Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers are commonly used in genetic analysis, microsatellites and SNPs.

Microsatellite: DNA of eukaryotic cells comprising a repetitive, short sequence of DNA that is present as tandem repeats and in highly variable copy number, flanked by sequences unique to that locus.

Mutant sequence: if it differs from one or more wild-type sequences. For example, a nucleic acid from a gene listed in Tables 2-4 containing a particular allele of a single nucleotide polymorphism may be a mutant sequence. In some cases, the individual carrying this allele has increased susceptibility toward the disorder or condition of interest. In other cases, the mutant sequence might also refer to an allele that decreases the susceptibility toward a disorder or condition of interest and thus acts in a protective manner. The term mutation may also be used to describe a specific allele of a polymorphic locus.

Non-conservative variants: are those in which a change in one or more nucleotides in a given codon position results in a polypeptide sequence in which a given amino acid residue in a polypeptide has been replaced by a non-conservative amino acid substitution. Non-conservative variants also include polypeptides comprising non-conservative amino acid substitutions.

Nucleic acid or polynucleotide: purine- and pyrimidine-containing polymers of any length, either polyribonucleotides or polydeoxyribonucleotide or mixed polyribo polydeoxyribonucleotides. This includes single- and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.

Nucleotide: a nucleotide, the unit of a DNA molecule, is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process.

Operably linked: means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

Ortholog: denotes a gene or polypeptide obtained from one species that has homology to an analogous gene or polypeptide from a different species.

Paralog: denotes a gene or polypeptide obtained from a given species that has homology to a distinct gene or polypeptide from that same species.

Phenotype: any visible, detectable or otherwise measurable property of an organism such as symptoms of, or susceptibility to, a disorder.

Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a disorder or condition of interest.

Portion and fragment: are synonymous. A portion as used with regard to a nucleic acid or polynucleotide refers to fragments of that nucleic acid or polynucleotide. The fragments can range in size from 8 nucleotides to all but one nucleotide of the entire gene sequence. Preferably, the fragments are at least about 8 to about 10 nucleotides in length; at least about 12 nucleotides in length; at least about 15 to about 20 nucleotides in length; at least about 25 nucleotides in length; or at least about 35 to about 55 nucleotides in length.

Probe or primer: refers to a nucleic acid or oligonucleotide that forms a hybrid structure with a sequence in a target region of a nucleic acid due to complementarity of the probe or primer sequence to at least one portion of the target region sequence.

Protein and polypeptide: are synonymous. Peptides are defined as fragments or portions of polypeptides, preferably fragments or portions having at least one functional activity (e.g., proteolysis, adhesion, fusion, antigenic, or intracellular activity) as the complete polypeptide sequence.

Recombinant nucleic acids: nucleic acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.

Regulatory sequence: refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

Sample: as used herein refers to a biological sample, such as, for example, tissue or fluid isolated from an individual or animal (including, without limitation, plasma, serum, cerebrospinal fluid, lymph, tears, nails, hair, saliva, milk, pus, and tissue exudates and secretions) or from in vitro cell culture-constituents, as well as samples obtained from, for example, a laboratory procedure.

Single nucleotide polymorphism (SNP): variation of a single nucleotide. This includes the replacement of one nucleotide by another and deletion or insertion of a single nucleotide. Typically, SNPs are biallelic markers although tri- and tetra-allelic markers also exist. For example, SNP MC may comprise allele C or allele A (Tables 5-37). Thus, a nucleic acid molecule comprising SNP A\C may include a C or A at the polymorphic position. For clarity purposes, an ambiguity code is used in Tables 5-37 and the sequence listing, to represent the variations. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another.

Sequence-conservative: variants are those in which a change of one or more nucleotides in a given codon position results in no alteration in the amino acid encoded at that position (i.e., silent mutation).

Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90% (M. Kanehisa, 1984, Nucl. Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

Wild-type gene from Tables 2-4: refers to the reference sequence. The wild-type gene sequences from Tables 2-4 used to identify the variants (polymorphisms, alleles, and haplotypes) described in detail herein.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes 1 and 11; M. J. Gait (ed), 1984, Oligonucleotide Synthesis; B. D. Hames and S. J. Higgins (eds), 1984, Nucleic Acid Hybridization; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Intl Publishers Ltd., Publ., London; D. P. Stites and A. L Terr, 1991, Basic and Clinical Immunology, 7th Ed., Appleton & Lange, Publ., Norwalk, Conn.; and A. K: Abbas et al., 1991, Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference is made in the following description and examples are generally obtainable from commercial sources, and specific vendors are cited herein.

### DETAILED DESCRIPTION OF THE INVENTION

### General Description of ADHD Disease

Children with attention deficit/hyperactivity disorder (ADHD) show signs of excessively high activity levels, restlessness, impulsivity and inattention. In Canada, it is estimated to occur in 2% to 12% of children, with an over-representation of boys by approximately 3:1 (Boyle et al., 1993; Offord et al., 1987; Tannock, 1998). Children with ADHD have difficulties listening to instructions, organizing their work, finishing schoolwork or chores, engaging in tasks that require sustained mental effort, engaging in quiet activities, sitting still, or waiting their turn. These problems are present before the age of 7 years and, in most cases, diagnosis will be made when starting primary school.

There is no single definitive test for the diagnosis of ADHD. However, The American Psychiatric Association has set up a number of criteria for the diagnosis of ADHD (Diagnostic and Statistical Manual of Mental Disorders DSM-IV et DSM-IVR: American Psychiatric Association, 1994 and 2000). The disease can be subdivided into three different subtypes:
1. Attention-deficit/hyperactivity disorder, combined type
2. Attention-deficit/hyperactivity disorder, predominantly inattentive type
3. Attention-deficit/hyperactivity disorder, predominantly hyperactive-impulsive type

Inattention:
a. often fails to give close attention to details or makes careless mistakes in schoolwork, work, or other activities
b. often has difficulty sustaining attention in tasks or play activities
c. often does not seem to listen when spoken to directly
d. often does not follow through on instructions and fails to finish schoolwork, chores, or duties in the workplace (not due to oppositional behavior or failure to understand instructions)
e. often has difficulty organizing tasks and activities
f. often avoids, dislikes, or is reluctant to engage in tasks that require sustained mental effort (such as schoolwork or homework)
g. often loses things necessary for tasks or activities (e.g., toys, school assignments, pencils, books, or tools)
h. is often easily distracted by extraneous stimuli
i. is often forgetful in daily activities

Hyperactivity
a. often fidgets with hands or feet or squirms in seat
b. often leaves seat in classroom or in other situations in which remaining seated is expected
c. often runs about or climbs excessively in situations in which it is inappropriate (in adolescents or adults, may be limited to subjective feelings of restlessness)
d. often has difficulty playing or engaging in leisure activities quietly
e. is often “on the go” or often acts as if “driven by a motor”
f. often talks excessively

Impulsivity
g. often blurts out answers before questions have been completed
h. often has difficulty awaiting turn
i. often interrupts or intrudes on others (e.g., butts into conversations or games)

ADHD diagnosis is made only when the child shows either six (6) or more of the symptoms of inattention OR six (6) or more of the symptoms of hyperactivity-impulsivity OR six (6) symptoms of each category for the combined type. Those symptoms have persisted for at least 6 months to a degree that is maladaptive and inconsistent with developmental level of a child that age.

ADHD incidence is observed more in boys than girls; the male-to-female ratios ranging from 3:1 and 9:1 (Fergusson & Horwood, 1993; McDermott, 1996; Valla et al., 1994). However, girls seem to have the inattentive type of ADHD more often, and may thus not be properly diagnosed. Thus the discrepancy in ratios between the sexes may be because many girls are under-diagnosed (Hudziak et al., 1998; NIH Consensus report, 2000). However, boys with the Predominantly Inattentive Type also tend to be under-diagnosed, so that argument alone cannot explain the gender difference.

ADHD symptoms can persist into adolescence and adulthood which results in difficulties in occupational, social and family lives. They have social difficulties, and they often end up engaging in antisocial activities such as drug and alcohol abuse (Murphy, 2002), and criminal activities and drop out of school (Faraone & Biederman, 1998; Modigh et al., 1998). They are also more prone to risk taking which makes them more susceptible to injuries. In addition, families with children with ADHD will often come under tremendous stress, including increased levels of parental frustration, and higher rates of divorce (NIH Consensus report, 2000). Furthermore, and considering the familial incidence of the disorder, the parent may himself have to face problems related to ADHD. However, it has been suggested that up to 50% of the cases still suffer from disabling symptoms at age 20 (Modigh et al., 1998; Spencer et al., 1998). ADHD might even be the most common undiagnosed psychiatric disorder in adults (Wender, 1998).

Neurophysiological studies of individuals with ADHD suggest that either the frontal cortex of the brain is dysfunctional, or there is some subcortical projection making it look as if the front is malfunctioning. Structural imaging studies of the brains of patients with ADHD have revealed damage to the brain, consistent with the fronto-subcortical classification (Biederman & Spencer, 1999; Ernst et al., 1998). The fronto-subcortical systems which control attention and motor behavior are rich in catecholamines. This is of particular interest, since many of the pharmaceuticals used for treating ADHD interfere with the catecholamine balance (Wilens, 2006).

Non-surgical treatment for active disease involves the use of stimulant drugs, i.e. methylphendiate (Ritalin®) and dextroamphetamine (Dexedrine®), where methylphendiate has been promoted more extensively by the drug industry, studied more often, and therefore are more widely prescribed (Elia et al., 1999). Both Ritalin® and Dexedrine® have similar side effects, and have been shown to be effective in children as well as in adults. No studies are available where children on medication have been followed into adulthood. Although drugs improve the abilities to do usual tasks in schoolwork, there has been no improvement in long-term academic achievement (Williams et al., 1999). Children who have other learning disabilities as well as ADHD may not respond so well to the stimulant drugs.

There have been several family studies (Biederman et al., 1990; Faraone et al., 1996; Gross-Tsur et al., 1991) or studies on girls (Faraone et al., 1991) as well as studies on African-American children (Samuel et al., 1999) that all show that there is a strong genetic component to ADHD. Segregation analysis suggested that the sex-dependent Mendelian codominant model best supported the data (Maher et al., 1999).

Twin studies as reviewed by Thapar et al. 1999 and Tannock 1998 show heritability estimates from 0.39 to 0.91. The studies on twins were largely carried out as interviews with mothers and or teachers. There is some bias in using the mothers as reporters, therefore it is important to use an impartial source as well (Sherman et al., 1997). This seems to be especially important for dizygotic twins where the behaviour of one twin has an inhibitory influence on the other, or where there is a maternal contrast effect (Thapar et al., 1999).

There have been only three whole-genome linkage studies: two affected sib pair (ASP) linkage studies (Ogdie et al., 2003 and Bakker et al., 2003) from the USA and the Netherlands and one study of multiplex families from Colombia (Arcos-Burgos et al., 2004). In the Dutch study of 164 ASPs, two regions on chromosomes 7p and 15q showed suggestive evidence of linkage (Bakker et al., 2003). The US (UCLA) study on 270 ASPs demonstrated significance for the chromosomal regions 16p13 and 17 μl. Parametric linkage analysis on the combined set of families of 16 multigenerational and extended pedigrees from Colombia showed significance on chromosomes 5q33.3, 11q22 and 17 μl (Arcos-Burgos et al., 2004). Fine mapping linkage analysis of all families together yielded significant linkage at chromosomes 4q13.2, 5q33, 3, 11q22 and 17 μl (Arcos-Burgos et al., 2004).

Thus the discovery of more disease genes and the development of GeneMaps for ADHD may lead to a better understanding of pathogenesis and to the identification of new pathways and genetic interactions involved in the disease, ultimately leading to better treatments for the patients. GeneMaps may also lead to molecular diagnostic tools that will identify subjects with ADHD or at risk for ADHD or for any related subtypes of the disease.

### Genome Wide Association Study to Construct a GeneMap for ADHD

The present invention is based on the discovery of genes associated with ADHD disease. In the preferred embodiment, disease-associated loci (candidate regions; Table 1) are identified by the statistically significant differences in allele or haplotype frequencies between the cases and the controls. For the purpose of the present invention, candidate regions (Table 1) are identified.

The invention provides a method for the discovery of genes associated with ADHD disease and the construction of a GeneMap for ADHD disease in a human population, comprising the following steps (see also Example section herein):

Step 1: Recruit Patients (Cases) and Controls

In the preferred embodiment, 500 patients diagnosed for ADHD disease along with two family members are recruited from the Quebec Founder Population (QFP). The preferred trios recruited are parent-parent-child (PPC) trios. Trios can also be recruited as parent-child-child (PCC) trios. In another preferred embodiment, more or less than 500 trios are recruited. In another embodiment, independent case and control samples are recruited.

In another embodiment, the present invention is performed as a whole or partially with DNA samples from individuals of another founder population than the Quebec population or from the general population.

Step 2: DNA Extraction and Quantitation

Any sample comprising cells or nucleic acids from patients or controls may be used. Preferred samples are those easily obtained from the patient or control. Such samples include, but are not limited to blood, peripheral lymphocytes, buccal swabs, epithelial cell swabs, nails, hair, bronchoalveolar lavage fluid, sputum, or other body fluid or tissue obtained from an individual.

In one embodiment, DNA is extracted from such samples in the quantity and quality necessary to perform the invention using conventional DNA extraction and quantitation techniques. The present invention is not linked to any DNA extraction or quantitation platform in particular.

Step 3: Genotype the Recruited Individuals

In one embodiment, assay-specific and/or locus-specific and/or allele-specific oligonucleotides for every SNP marker of the present invention (Tables 5-37) are organized onto one or more arrays. The genotype at each SNP locus is revealed by hybridizing short PCR fragments comprising each SNP locus onto these arrays. The arrays permit a high-throughput genome wide association study using DNA samples from individuals of the Quebec founder population. Such assay-specific and/or locus-specific and/or allele-specific oligonucleotides necessary for scoring each SNP of the present invention are preferably organized onto a solid support. Such supports can be arrayed on wafers, glass slides, beads or any other type of solid support.

In another embodiment, the assay-specific and/or locus-specific and/or allele-specific oligonucleotides are not organized onto a solid support but are still used as a whole, in panels or one by one. The present invention is therefore not linked to any genotyping platform in particular.

In another embodiment, one or more portions of the SNP maps (publicly available maps and our own proprietary QLDM map) are used to screen the whole genome, a subset of chromosomes, a chromosome, a subset of genomic regions or a single genomic region.

In the preferred embodiment, the individuals composing the 500 trios or the cases and controls are preferably individually genotyped with at least 80,000 markers, generating at least a few million genotypes; more preferably, at least a hundred million. In another embodiment, individuals are pooled in cases and control pools for genotyping and genetic analysis.

Step 4: Exclude the Markers that Did not Pass the Quality Control of the Assay.

Preferably, the quality controls comprises, but are not limited to, the following criteria: eliminate SNPs that had a high rate of Mendelian errors (cut-off at 1% Mendelian error rate), that deviate from the Hardy-Weinberg equilibrium, that are non-polymorphic in the Quebec founder population or have too many missing data (cut-off at 1% missing values or higher), or simply because they are non-polymorphic in the Quebec founder population (cut-off at 1%≦10% minor allele frequency (MAF)).

Step 5: Perform the Genetic Analysis on the Results Obtained Using Haplotype Information as Well as Single-Marker Association.

In the preferred embodiment, genetic analysis is performed on all the genotypes from Step 3.

In another embodiment, genetic analysis is performed on a subset of markers from Step 3 or from markers that passed the quality controls from Step 4.

In one embodiment, the genetic analysis consists of, but is not limited to features corresponding to Phase information and haplotype structures. Phase information and haplotype structures are preferably deduced from trio genotypes using Phasefinder. Since chromosomal assignment (phase) cannot be estimated when all trio members are heterozygous, an Expectation-Maximization (EM) algorithm may be used to resolve chromosomal assignment ambiguities after Phasefinder.

In yet another embodiment, the PL-EM algorithm (Partition-Ligation EM; Niu et al., Am. J. Hum. Genet. 70:157 (2002)) can be used to estimate haplotypes from the “genotype” data as a measured estimate of the reference allele frequency of a SNP in 15-marker windows that advance in increments of one marker across the data set. The results from such algorithms are converted into 15-marker haplotype files. Subsequently, the individual 15-marker block files are assembled into one continuous block of haplotypes for the entire chromosome. These extended haplotypes can then be used for further analysis. Such haplotype assembly algorithms take the consensus estimate of the allele call at each marker over all separate estimations (most markers are estimated 15 different times as the 15 marker blocks pass over their position).

In the preferred embodiment, the haplotypes for both the controls and the patients are derived in this manner. The preferred control of a trio structure is the non-transmitted chromosomes (chromosomes found in parents but not in affected child) if the patient is the child.

In another embodiment, the haplotype frequencies among patients are compared to those among the controls using LDSTATS, a program that assesses the association of haplotypes with the disease. Such program defines haplotypes using multi-marker windows that advance across the marker map in one-marker increments. Such windows can be 1, 3, 5, 7 or 9 markers wide, and all these window sizes are tested concurrently. Larger multi-marker haplotype windows can also be used. At each position the frequency of haplotypes in cases is compared to the frequency of haplotypes in controls. Such allele frequency differences for single marker windows can be tested using Pearson's Chi-square with any degree of freedom. Multi-allelic haplotype association can be tested using Smith's normalization of the square root of Pearson's Chi-square. Such significance of association can be reported in two ways:

The significance of association within any one haplotype window is plotted against the marker that is central to that window.

P-values of association for each specific marker are calculated as a pooled P-value across all haplotype windows in which they occur. The pooled P-value is calculated using an expected value and variance calculated using a permutation test that considers covariance between individual windows. Such pooled P-values can yield narrower regions of gene location than the window data (see example 3 for details on analysis methods, such as LDSTATS v2.0 and v4.0).

In another embodiment, conditional haplotype and subtype analyses can be performed on subsets of the original set of cases and controls using the program LDSTATS. For conditional analyses, the selection of a subset of cases and their matched controls can be based on the carrier status of cases at a gene or locus of interest (see conditional analysis section in example 3 herein). Various conditional haplotypes can be derived, such as protective haplotypes and risk haplotypes.

Step 6: SNP and DNA Polymorphism Discovery

In the preferred embodiment, all the candidate genes and regions identified in step 5 are sequenced for polymorphism identification.

In another embodiment, the entire region, including all introns, is sequenced to identify all polymorphisms.

In yet another embodiment, the candidate genes are prioritized for sequencing, and only functional gene elements (promoters, conserved noncoding sequences, exons and splice sites) are sequenced.

In yet another embodiment, previously identified polymorphisms in the candidate regions can also be used. For example, SNPs from dbSNP, or others can also be used rather than resequencing the candidate regions to identify polymorphisms.

The discovery of SNPs and DNA polymorphisms generally comprises a step consisting of determining the major haplotypes in the region to be sequenced. The preferred samples are selected according to which haplotypes contribute to the association signal observed in the region to be sequenced. The purpose is to select a set of samples that covers all the major haplotypes in the given region. Each major haplotype is preferably analyzed in at least a few individuals.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Any means of mutation detection or discrimination may be used. For instance, DNA sequencing, scanning methods, hybridization, extension based methods, incorporation based methods, restriction enzyme-based methods and ligation-based methods may be used in the methods of the invention.

Sequencing methods include, but are not limited to, direct sequencing, and sequencing by hybridization. Scanning methods include, but are not limited to, protein truncation test (PTT), single-strand conformation polymorphism analysis (SSCP), denaturing gradient gel electrophoresis (DGGE), temperature gradient gel electrophoresis (TGGE), cleavage, heteroduplex analysis, chemical mismatch cleavage (CMC), and enzymatic mismatch cleavage. Hybridization-based methods of detection include, but are not limited to, solid phase hybridization such as dot blots, multiple allele specific diagnostic assay (MASDA), reverse dot blots, and oligonucleotide arrays (DNA Chips). Solution phase hybridization amplification methods may also be used, such as Taqman. Extension based methods include, but are not limited to, amplification refraction mutation systems (ARMS), amplification refractory mutation systems (ALEX), and competitive oligonucleotide priming systems (COPS). Incorporation based methods include, but are not limited to, mini-sequencing and arrayed primer extension (APEX). Restriction enzyme-based detection systems include, but are not limited to, restriction site generating PCR. Lastly, ligation based detection methods include, but are not limited to, oligonucleotide ligation assays (OLA). Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, colometric methods, hybridization protection assays and mass spectrometry methods. Further amplification methods include, but are not limited to self sustained replication (SSR), nucleic acid sequence based amplification (NASBA), ligase chain reaction (LCR), strand displacement amplification (SDA) and branched DNA (B-DNA).

Sequencing can also be performed using a proprietary sequencing technology (Cantaloupe; PCT/EP2005/002870).

Step 7: Ultrafine Mapping

This step further maps the candidate regions and genes confirmed in the previous step to identify and validate the responsible polymorphisms associated with ADHD disease in the human population.

In a preferred embodiment, the discovered SNPs and polymorphisms of step 6 are ultrafine mapped at a higher density of markers than the GWS described herein using the same technology described in step 3.

Step 8: GeneMap Construction

The confirmed variations in DNA (including both genic and non-genic regions) are used to build a GeneMap for ADHD disease. The gene content of this GeneMap is described in more detail below. Such GeneMap can be used for other methods of the invention comprising the diagnostic methods described herein, the susceptibility to ADHD disease, the response to a particular drug, the efficacy of a particular drug, the screening methods described herein and the treatment methods described herein.

As is evident to one of ordinary skill in the art, all of the above steps or the steps do not need to be performed, or performed in a given order to practice or use the SNPs, genomic regions, genes, proteins, etc. in the methods of the invention.

Genes from the GeneMap

In one embodiment the GeneMap consists of genes and targets, in a variety of combinations, identified from the candidate regions listed in Table 1. In another embodiment, all genes from Tables 2-4 are present in the GeneMap. In another preferred embodiment, the GeneMap consists of a selection of genes from Tables 2-4. The genes of the invention (Tables 2-4) are arranged by candidate regions and by their chromosomal location. Such order is for the purpose of clarity and does not reflect any other criteria of selection in the association of the genes with ADHD disease.

In one embodiment, genes identified in the WGAS and subsequent studies are evaluated using the Ingenuity Pathway Analysis application (IPA, Ingenuity systems) in order to identify direct biological interactions between these genes, and also to identify molecular regulators acting on those genes (indirect interactions) that could be also involved in ADHD. The purpose of this effort is to decipher the molecules involved in contributing to ADHD. These gene interaction networks are very valuable tools in the sense that they facilitate extension of the map of gene products that could represent potential drug targets for ADHD.

In another embodiment, other means (such as functional biochemical assays and genetic assays) are used to identify the biological interactions between genes to create a GeneMap.

In yet another embodiment, the GeneMaps of the invention consists of a selection of genes from Tables 2-4 and a selection of genes that are interactors (direct or indirect) with the genes from the Tables. For clarity purposes, those interactor genes are not present in Tables 2-4, but know in the art from various public documents (scientific articles, patent literature etc.).

The GeneMaps aid in the selection of the best target to intervene in a disease state. Each disease can be subdivided into various disease states and sub-phenotypes, thus various GeneMaps are needed to address various disease sub-phenotypes, and a clinical population can be stratified by sub-phenotype, which would be covered by a particular GeneMap.

### Nucleic Acid Sequences

The nucleic acid sequences of the present invention may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, nongenic regions, and regulatory regions. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means. The nucleic acids described herein are used in certain embodiments of the methods of the present invention for production of RNA, proteins or polypeptides, through incorporation into cells, tissues, or organisms. In one embodiment, DNA containing all or part of the coding sequence for the genes described in Tables 2-4, or the SNP markers described in Tables 5-37, is incorporated into a vector for expression of the encoded polypeptide in suitable host cells. The invention also comprises the use of the nucleotide sequence of the nucleic acids of this invention to identify DNA probes for the genes described in Tables 2-4 or the SNP markers described in Tables 5-37, PCR primers to amplify the genes described in Tables 2-4 or the SNP markers described in Tables 5-37, nucleotide polymorphisms in the genes described in Tables 2-4, and regulatory elements of the genes described in Tables 2-4. The nucleic acids of the present invention find use as primers and templates for the recombinant production of ADHD disease-associated peptides or polypeptides, for chromosome and gene mapping, to provide antisense sequences, for tissue distribution studies, to locate and obtain full length genes, to identify and obtain homologous sequences (wild-type and mutants), and in diagnostic applications.

### Antisense Oligonucleotides

In a particular embodiment of the invention, an antisense nucleic acid or oligonucleotide is wholly or partially complementary to, and can hybridize with, a target nucleic acid (either DNA or RNA) having the sequence of SEQ ID NO:1, NO:3 or any SEQ ID from any Tables of the invention. For example, an antisense nucleic acid or oligonucleotide comprising 16 nucleotides can be sufficient to inhibit expression of at least one gene from Tables 2-4. Alternatively, an antisense nucleic acid or oligonucleotide can be complementary to 5′ or 3′ untranslated regions, or can overlap the translation initiation codon (5′ untranslated and translated regions) of at least one gene from Tables 2-4, or its functional equivalent. In another embodiment, the antisense nucleic acid is wholly or partially complementary to, and can hybridize with, a target nucleic acid that encodes a polypeptide from a gene described in Tables 2-4.

In addition, oligonucleotides can be constructed which will bind to duplex nucleic acid (i.e., DNA:DNA or DNA:RNA), to form a stable triple helix containing or triplex nucleic acid. Such triplex oligonucleotides can inhibit transcription and/or expression of a gene from Tables 2-4, or its functional equivalent (M. D. Frank-Kamenetskii et al., 1995). Triplex oligonucleotides are constructed using the basepairing rules of triple helix formation and the nucleotide sequence of the genes described in Tables 2-4.

The present invention encompasses methods of using oligonucleotides in antisense inhibition of the function of the genes from Tables 2-4. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention. Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH₃, F, OCH3, OCN, O(CH2), NH2 and O(CH2)nCH3, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one gene from Tables 2-4 DNA or RNA to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds.

Antisense nucleic acids or oligonulcleotides can be produced by standard techniques (see, e.g., Shewmaker et al., U.S. Pat. No. 6,107,065). The oligonucleotides used in accordance with this invention may be conveniently and routinely made through the well-known technique of solid phase synthesis. Any other means for such synthesis may also be employed; however, the actual synthesis of the oligonucleotides is well within the abilities of the practitioner. It is also well known to prepare other oligonucleotides such as phosphorothioates and alkylated derivatives.

The oligonucleotides of this invention are designed to be hybridizable with RNA (e.g., mRNA) or DNA from genes described in Tables 2-4. For example, an oligonucleotide (e.g., DNA oligonucleotide) that hybridizes to mRNA from a gene described in Tables 2-4 can be used to target the mRNA for RnaseH digestion. Alternatively an oligonucleotide that can hybridize to the translation initiation site of the mRNA of a gene described in Tables 2-4 can be used to prevent translation of the mRNA. In another approach, oligonucleotides that bind to the double-stranded DNA of a gene from Tables 2-4 can be administered. Such oligonucleotides can form a triplex construct and inhibit the transcription of the DNA encoding polypeptides of the genes described in Tables 2-4. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; 3′ coding region; DNA replication initiation and elondation sites. Preferably, the complementary oligonucleotide is designed to hybridize to the most unique 5′ sequence of a gene described in Tables 2-4, including any of about 15-35 nucleotides spanning the 5′ coding sequence. In accordance with the present invention, the antisense oligonucleotide can be synthesized, formulated as a pharmaceutical composition, and administered to a subject. The synthesis and utilization of antisense and triplex oligonucleotides have been previously described (e.g., Simon et al., 1999; Barre et al., 2000; Elez et al., 2000; Sauter et al., 2000).

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a polypeptide from the genes described in Tables 2-4. These techniques are described both in Sambrook et al., 1989 and in Ausubel et al., 1992. For example, expression of at least one gene from Tables 2-4 can be inhibited by transforming a cell or tissue with an expression vector that expresses high levels of untranslatable sense or antisense sequences. Even in the absence of integration into the DNA, such vectors may continue to transcribe RNA molecules until they are disabled by endogenous nucleases. Transient expression may last for a month or more with a nonreplicating vector, and even longer if appropriate replication elements are included in the vector system. Various assays may be used to test the ability of gene-specific antisense oligonucleotides to inhibit the expression of at least one gene from Tables 2-4. For example, mRNA levels of the genes described in Tables 2-4 can be assessed by Northern blot analysis (Sambrook et al., 1989; Ausubel et al., 1992; J. C. Alwine et al. 1977; I. M. Bird, 1998), quantitative or semi-quantitative RT-PCR analysis (see, e.g., W. M. Freeman et al., 1999; Ren et al., 1998; J. M. Cale et al., 1998), or in situ hybridization (reviewed by A. K. Raap, 1998). Alternatively, antisense oligonucleotides may be assessed by measuring levels of the polypeptide from the genes described in Tables 2-4, e.g., by western blot analysis, indirect immunofluorescence and immunoprecipitation techniques (see, e.g., J. M. Walker, 1998, Protein Protocols on cD-ROM, Humana Press, Totowa, N.J.). Any other means for such detection may also be employed, and is well within the abilities of the practitioner.

### Mapping Technologies

The present invention includes various methods which employ mapping technologies to map SNPs and polymorphisms. For purpose of clarity, this section comprises, but is not limited to, the description of mapping technologies that can be utilized to achieve the embodiments described herein. Mapping technologies may be based on amplification methods, restriction enzyme cleavage methods, hybridization methods, sequencing methods, and cleavage methods using agents.

Amplification methods include: self sustained sequence replication (Guatelli et al., 1990), transcriptional amplification system (Kwoh et al., 1989), Q-Beta Replicase (Lizardi at al., 1988), isothermal amplification (e.g. Dean at al., 2002; and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

Restriction enzyme cleavage methods include: isolating sample and control DNA, amplification (optional), digestion with one or more restriction endonucleases, determination of fragment length sizes by gel electrophoresis and comparing samples and controls. Differences in fragment length sizes between sample and control DNA indicates mutations in the sample DNA. Moreover, sequence specific ribozymes (see, e.g., U.S. Pat. No. 5,498,531 or DNAzyme e.g. U.S. Pat. No. 5,807,718) can be used to score for the presence of specific mutations by development or loss of a ribozyme or DNAzyme cleavage site.

Hybridization methods include any measurement of the hybridization or gene expression levels, of sample nucleic acids to probes corresponding to about 2, 3, 4, 5, 6, 7, 8, 9, 10, 15, 20, 25, 30, 50, 75, 100, 200, 500, 1000 or more genes, or ranges of these numbers, such as about 5-20, about 10-20, about 20-50, about 50-100, or about 100-200 genes of Tables 2-4.

SNPs and SNP maps of the invention can be identified or generated by hybridizing sample nucleic acids, e.g., DNA or RNA, to high density arrays or bead arrays containing oligonucleotide probes corresponding to the polymorphisms of Tables 5-37 (see the Affymetrix arrays and Illumina bead sets at www.affymetrix.com and www.illumina.com and see Cronin et al., 1996; or Kozal et al., 1996).

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface precedes using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of, hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency to ensure hybridization and then subsequent washes are performed at higher stringency to eliminate mismatched hybrid duplexes. Successive washes may be performed at increasingly higher stringency until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes or gene fragments described in Tables 2-4 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes (see GeneChip® Expression Analysis Manual, Affymetrix, Rev. 3, which is herein incorporated by reference in its entirety).

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

A variety of sequencing reactions known in the art can be used to directly sequence nucleic acids for the presence or the absence of one or more polymorphisms of Tables 5-37. Examples of sequencing reactions include those based on techniques developed by Maxam and Gilbert (1977) or Sanger (1977). It is also contemplated that any of a variety of automated sequencing procedures can be utilized, including sequencing by mass spectrometry (see, e.g. PCT International Publication No. WO 94/16101; Cohen et al., 1996; and Griffin et al., 1993), real-time pyrophosphate sequencing method (Ronaghi et al., 1998; and Permutt et al., 2001) and sequencing by hybridization (see e.g. Drmanac et al., 2002).

Other methods of detecting polymorphisms include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing a wild-type sequence with potentially mutant RNA or DNA obtained from a sample. The double-stranded duplexes are treated with an agent who cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of a mutation or SNP (see, for example, Cotton et al., 1988; and Saleeba et al., 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping polymorphisms. For example, the mutY enzyme of E. coli cleaves A at G/A mismatches (Hsu et al., 1994). Other examples include, but are not limited to, the MutHLS enzyme complex of E. coli (Smith and Modrich Proc. 1996) and Cel 1 from the celery (Kulinski et al., 2000) both cleave the DNA at various mismatches. According to an exemplary embodiment, a probe based on a polymorphic site corresponding to a polymorphism of Tables 5-37 is hybridized to a cDNA or other DNA product from a test cell or cells. The duplex is treated with a DNA mismatch repair enzyme, and the cleavage products, if any, can be detected from electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify polymorphisms in a sample. For example, single strand conformation polymorphism (SSCP) analysis can be used to detect differences in electrophoretic mobility between mutant and wild type nucleic acids (Orita et al., 1989; Cotton et al., 1993; and Hayashi 1992). Single-stranded DNA fragments of case and control nucleic acids will be denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et a, 1985). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoom et al., 2000).

Examples of other techniques for detecting polymorphisms include, but are not limited to, selective oligonucleotide hybridization, selective amplification, selective primer extension, selective ligation, single-base extension, selective termination of extension or invasive cleavage assay. For example, oligonucleotide primers may be prepared in which the polymorphism is placed centrally and then hybridized to target DNA under conditions which permit hybridization only if a perfect match is found (Saiki et al., 1986; Saiki et al., 1989). Such oligonucleotides are hybridized to PCR amplified target DNA or a number of different mutations when the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

Hybridization assays may also be carried out with a temperature gradient following the principle of dynamic allele-specific hybridization or like e.g. Jobs et al., (2003); and Bourgeois and Labuda, (2004). For example, the hybridization is done using one of the two allele-specific oligonucleotides labeled with a fluorescent dye, and an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of an associated allele, a particular allele of a polymorphic locus, or the like (e.g. Jobs et al., 2003). Alternatively, the hybridization is done under a gradually decreasing temperature. In this case, both allele-specific oligonucleotides are hybridized to the template competitively. At high temperature none of the two probes are hybridized. Once the optimal temperature of the full-matched probe is reached, it hybridizes and leaves no target for the mismatched probe (e.g. Bourgeois and Labuda, 2004). In the latter case, if the allele-specific probes are differently labeled, then they are hybridized to a single PCR-amplified target. If the probes are labeled with the same dye, then the probe cocktail is hybridized twice to identical templates with only one labeled probe, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, a particular allele of a polymorphic locus, or the like, also referred to as “mutation” of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the mutation to create cleavage-based detection (Gasparini et al., 1992). It is anticipated that in certain embodiments, amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNPs and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, and bead array or like. This reaction can be performed at a high multiplexed level.

In another alternative, the associated allele, a particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, and bead array or like. In the latter case, the extension oligonucleotide will contain a SNP-specific zip code tag.

In yet another alternative, a SNP is scored by selective termination of extension. The template is first amplified by PCR and the extension oligonucleotide hybridizes in the vicinity of the SNP locus, close to but not necessarily adjacent to it. The extension reaction is carried out using a thermostable polymerase such as ThermoSequenase (GE Healthcare) in the presence of a mix of dNTPs and at least one ddNTP. The latter has to terminate the extension at one of the allele of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, SNPs are detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Methods to Identify Agents that Modulate the Expression of a Nucleic Acid Encoding a Gene Involved in ADHD

The present invention provides methods for identifying agents that modulate the expression of a nucleic acid encoding a gene from Tables 2-4. Such methods may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Such cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: muscle cells, nervous cells, blood and vessels cells, T cell, mast cell, lymphocyte, monocyte, macrophage, and epithelial cells.

In one assay format, the expression of a nucleic acid encoding a gene of the invention (see Tables 2-4) in a cell or tissue sample is monitored directly by hybridization to the nucleic acids of the invention. Cell lines or tissues are exposed to the agent to be tested under appropriate conditions and time and total RNA or mRNA is isolated by standard procedures such as those disclosed in Sambrook et al., (1989) Molecular Cloning: A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook et al., and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

Methods to Identify Agents that Modulate the Activity of a Protein Encoded by a Gene Involved in ADHD Disease and Antibodies of the Invention

The present invention provides methods for identifying agents that modulate at least one activity of the proteins described in Tables 2-4. Such methods may utilize any means of monitoring or detecting the desired activity. As used herein, an agent is said to modulate the expression of a protein of the invention if it is capable of up- or down-regulating expression of the protein in a cell. Such cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: muscle cells, nervous cells, blood and vessels cells, T cell, mast cell, lymphocyte, monocyte, macrophage, and epithelial cells.

In one format, the specific activity of a protein of the invention, normalized to a standard unit, may be assayed in a cell population that has been exposed to the agent to be tested and compared to an unexposed control cell population. Cell lines or populations are exposed to the agent to be tested under appropriate conditions and times. Cellular lysates may be prepared from the exposed cell line or population and a control, unexposed cell line or population. The cellular lysates are then analyzed with a probe, such as an antibody probe.

Antibodies and Antibody probes can be prepared by immunizing suitable mammalian hosts (e.g. mice or transgenic mice) utilizing appropriate immunization protocols using the proteins of the invention or antigen-containing fragments thereof. To enhance immunogenicity for immunization protocols, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin. The antibodies or fragments may also be produced, using current technology, by recombinant means. The antibody chains (light and heavy) may be cloned into the vector by methods known in the art. Specific antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Phage display techniques can be used to provide libraries containing a repertoire of antibodies with varying affinities for proteins, or fragments thereof, described in Tables 2-4. Techniques for the identification of high affinity human antibodies from such libraries are described by Griffiths et al., EMBO J., 13:3245-3260 (1994); Nissim et al., ibid, pp. 692-698 and by Griffiths et al., ibid, 12:725-734. The antibody of the invention also comprise humanized and human antibodies. Such antibodies are mage by methods known in the art.

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention includes antibodies or fragments thereof that bind to a protein encoded by a gene in Tables 2-4. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of the protein encoded by a gene from Tables 2-4. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

### Method to Diagnose ADHD

The present invention also relates to methods for diagnosing ADHD or a related disease, preferably a subtype of ADHD, a predisposition to such a disease and/or disease progression. In some methods, the steps comprise contacting a target sample with (a) nucleic acid molecule(s) or fragments thereof and comparing the concentration of individual mRNA(s) with the concentration of the corresponding mRNA(s) from at least one healthy donor. An aberrant (increased or decreased) mRNA level of at least one gene from Tables 2-4, at least 5 or 10 genes from Tables 2-4, at least 50 genes from Tables 2-4, at least 100 genes from Tables 2-4 or at least 200 genes from Tables 2-4 determined in the sample in comparison to the control sample is an indication of ADHD disease or a related subtype or a disposition to such kinds of diseases. For diagnosis, samples are, preferably, obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: muscle cells, nervous cells, blood and vessels cells, T cell, mast cell, lymphocyte, monocyte, macrophage, and epithelial cells.

For analysis of gene expression, total RNA is obtained from cells according to standard procedures and, preferably, reverse-transcribed. Preferably, a DNAse treatment (in, order to get rid of contaminating genomic DNA) is performed.

The nucleic acid molecule or fragment is typically a nucleic acid probe for hybridization or a primer for PCR. The person skilled in the art is in a position to design suitable nucleic acids probes based on the information provided in the Tables of the present invention. The target cellular component, i.e. mRNA, e.g., in brain tissue, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B10237362) and other detection assays that are known to those skilled in the art. Products obtained by in vitro amplification can be detected according to established methods, e.g. by separating the products on agarose or polyacrylamide gels and by subsequent staining with ethidium bromide or any other dye or reagent. Alternatively, the amplified products can be detected by using labeled primers for amplification or labeled dNTPs. Preferably, detection is based on a microarray.

The probes (or primers) (or, alternatively, the reverse-transcribed sample mRNAs) can be detectably labeled, for example, with a radioisotope, a bioluminescent compound, a chemiluminescent compound, a fluorescent compound, a metal chelate, or an enzyme.

The present invention also relates to the use of the nucleic acid molecules or fragments described above for the preparation of a diagnostic composition for the diagnosis of ADHD or a subtype or predisposition to such a disease.

The present invention also relates to the use of the nucleic acid molecules of the present invention for the isolation or development of a compound which is useful for therapy of ADHD. For example, the nucleic acid molecules of the invention and the data obtained using said nucleic acid molecules for diagnosis of ADHD might allow for the identification of further genes which are specifically dysregulated, and thus may be considered as potential targets for therapeutic interventions. Furthermore, such diagnostic might also be used for selection of patients that might respond positively or negatively to a potential target for therapeutic interventions (as for the pharmacogenomics and personalized medicine concept well know in the art; see prognostic assays text below).

The invention further provides prognostic assays that can be used to identify subjects having or at risk of developing ADHD. In such method, a test sample is obtained from a subject and the amount and/or concentration of the nucleic acid described in Tables 2-4 is determined; wherein the presence of an associated allele, a particular allele of a polymorphic locus, or the likes in the nucleic acids sequences of this invention (see SEQ ID from Tables 5-37) can be diagnostic for a subject having or at risk of developing ADHD. As used herein, a “test sample” refers to a biological sample obtained from a subject of interest. For example, a test sample can be a biological fluid, a cell sample, or tissue. A biological fluid can be, but is not limited to saliva, serum, mucus, urine, stools, spermatozoids, vaginal secretions, lymph, amniotic liquid, pleural liquid and tears. Cells can be, but are not limited to: hair cells, muscle cells, nervous cells, blood and vessels cells, dermis, epidermis and other skin cells, and various brain cells.

Furthermore, the prognostic assays described herein can be used to determine whether a subject can be administered an agent (e.g., an agonist, antagonist, peptidomimetic, polypeptide, nucleic acid such as antisense DNA or interfering RNA (RNAi), small molecule or other drug candidate) to treat ADHD. Specifically, these assays can be used to predict whether an individual will have an efficacious response or will experience adverse events in response to such an agent. For example, such methods can be used to determine whether a subject can be effectively treated with an agent that modulates the expression and/or activity of a gene from Tables 2-4 or the nucleic acids described herein. In another example, an association study may be performed to identify polymorphisms from Tables 5-37 that are associated with a given response to the agent, e.g., an efficacious response or the likelihood of one or more adverse events. Thus, one embodiment of the present invention provides methods for determining whether a subject can be effectively treated with an agent for a disease associated with aberrant expression or activity of a gene from Tables 2-4 in which a test sample is obtained and nucleic acids or polypeptides from Tables 2-4 are detected (e.g., wherein the presence of a particular level of expression of a gene from Tables 2-4 or a particular allelic variant of such gene, such as polymorphisms from Tables 5-37 is diagnostic for a subject that can be administered an agent to treat a disorder such as ADHD). In one embodiment, the method includes obtaining a sample from a subject suspected of having ADHD or an affected individual and exposing such sample to an agent. The expression and/or activity of the nucleic acids and/or genes of the invention are monitored before and after treatment with such agent to assess the effect of such agent. After analysis of the expression values, one skilled in the art can determine whether such agent can effectively treat such subject. In another embodiment, the method includes obtaining a sample from a subject having or susceptible to developing ADHD and determining the allelic constitution of polymorphisms from Tables 5-37 that are associated with a particular response to an agent. After analysis of the allelic constitution of the individual at the associated polymorphisms, one skilled in the art can determine whether such agent can effectively treat such subject.

The methods of the invention can also be used to detect genetic alterations in a gene from Tables 2-4, thereby determining if a subject with the lesioned gene is at risk for a disease associated with ADHD. In preferred embodiments, the methods include detecting, in a sample of cells from the subject, the presence or absence of a genetic alteration characterized by at least one alteration linked to or affecting the integrity of a gene from Tables 2-4 encoding a polypeptide or the misexpression of such gene. For example, such genetic alterations can be detected by ascertaining the existence of at least one of: (1) a deletion of one or more nucleotides from a gene from Tables 2-4; (2) an addition of one or more nucleotides to a gene from Tables 2-4; (3) a substitution of one or more nucleotides of a gene from Tables 2-4; (4) a chromosomal rearrangement of a gene from Tables 2-4; (5) an alteration in the level of a messenger RNA transcript of a gene from Tables 2-4; (6) aberrant modification of a gene from Tables 2-4, such as of the methylation pattern of the genomic DNA, (7) the presence of a non-wild type splicing pattern of a messenger RNA transcript of a gene from Tables 2-4; (8) inappropriate post-translational modification of a polypeptide encoded by a gene from Tables 2-4; and (9) alternative promoter use. As described herein, there are a large number of assay techniques known in the art which can be used for detecting alterations in a gene from Tables 2-4. A preferred biological sample is a peripheral blood sample obtained by conventional means from a subject. Another preferred biological sample is a buccal swab. Other biological samples can be, but are not limited to, urine, stools, spermatozoids, vaginal secretions, lymph, amniotic liquid, pleural liquid and tears.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or alternatively, in a ligation chain reaction (LCR) (see, e.g., Landegran et al., 1988; and Nakazawa et al., 1994), the latter of which can be particularly useful for detecting point mutations in a gene from Tables 2-4 (see Abavaya et al., 1995). This method can include the steps of collecting a sample of cells from a patient, isolating nucleic acid (e.g., genomic DNA, mRNA, or both) from the cells of the sample, contacting the nucleic acid sample with one or more primers which specifically hybridize to a gene from Tables 2-4 under conditions such that hybridization and amplification of the nucleic acid from Tables 2-4 (if present) occurs, and detecting the presence or absence of an amplification product, or detecting the size of the amplification product and comparing the length to a control sample. PCR and/or LCR may be desirable to use as a preliminary amplification step in conjunction with some of the techniques used for detecting a mutation, an associated allele, a particular allele of a polymorphic locus, or the like described in the above sections. Other mutation detection and mapping methods are described in previous sections of the detailed description of the present invention.

The present invention also relates to further methods for diagnosing ADHD or a related disorder or subtype, a predisposition to such a disorder and/or disorder progression. In some methods, the steps comprise contacting a target sample with (a) nucleic molecule(s) or fragments thereof and determining the presence or absence of a particular allele of a polymorphism that confers a disorder-related phenotype (e.g., predisposition to such a disorder and/or disorder progression). The presence of at least one allele from Tables 5-37 that is associated with ADHD (“associated allele”), at least 5 or 10 associated alleles from Tables 5-37, at least 50 associated alleles from Tables 5-37 at least 100 associated alleles from Tables 5-37, or at least 200 associated alleles from Tables 5-37 determined in the sample is an indication of ADHD disease or a related disorder, a disposition or predisposition to such kinds of disorders, or a prognosis for such disorder progression. Such samples and cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: muscle cells, nervous cells, blood and vessels cells, T cell, mast cell, lymphocyte, monocyte, macrophage, and epithelial cells.

In other embodiments, alterations in a gene from Tables 2-4 can be identified by hybridizing sample and control nucleic acids, e.g., DNA or RNA, to high density arrays or bead arrays containing tens to thousands of oligonucleotide probes (Cronin et al., 1996; Kozal et al., 1996). For example, alterations in a gene from Tables 2-4 can be identified in two dimensional arrays containing light-generated DNA probes as described in Cronin et al., (1996). Briefly, a first hybridization array of probes can be used to scan through long stretches of DNA in a sample and control to identify base changes between the sequences by making linear arrays of sequential overlapping probes. This step allows the identification of point mutations, associated alleles, particular alleles of a polymorphic locus, or the like. This step is followed by a second hybridization array that allows the characterization of specific mutations by using smaller, specialized probe arrays complementary to all variants, mutations, alleles detected. Each mutation array is composed of parallel probe sets, one complementary to the wild-type gene and the other complementary to the mutant gene.

In yet another embodiment, any of a variety of sequencing reactions known in the art can be used to directly sequence a gene from Tables 2-4 and detect an associated allele, a particular allele of a polymorphic locus, or the like by comparing the sequence of the sample gene from Tables 2-4 with the corresponding wild-type (control) sequence (see text described in previous sections for various sequencing techniques and other methods of detecting an associated allele, a particular allele of a polymorphic locus, or the likes in a gene from Tables 2-4. Such methods include methods in which protection from cleavage agents is used to detect mismatched bases in RNA/RNA, DNA/DNA or RNA/DNA heteroduplexes (Myers et al., 1985) and alterations in electrophoretic mobility. Examples of other techniques for detecting point mutations, an associated allele, a particular allele of a polymorphic locus, or the like include, but are not limited to, selective oligonucleotide hybridization, selective amplification, selective primer extension, selective ligation, single-base extension, selective termination of extension or invasive cleavage assay.

Other types of markers can also be used for diagnostic purposes. For example, microsatellites can also be useful to detect the genetic predisposition of an individual to a given disorder. Microsatellites consist of short sequence motifs of one or a few nucleotides repeated in tandem. The most common motifs are polynucleotide runs, dinucleotide repeats (particularly the CA repeats) and trinucleotide repeats. However, other types of repeats can also be used. The microsatellites are very useful for genetic mapping because they are highly polymorphic in their length. Microsatellite markers can be typed by various means, including but not limited to DNA fragment sizing, oligonucleotide ligation assay and mass spectrometry. For example, the locus of the microsatellite is amplified by PCR and the size of the PCR fragment will be directly correlated to the length of the microsatellite repeat. The size of the PCR-fragment can be detected by regular means of gel electrophoresis. The fragment can be labeled internally during PCR or by using end-labeled oligonucleotides in the PCR reaction (e.g. Mansfield et al., 1996). Alternatively, the size of the PCR fragment is determined by mass spectrometry. In another alternative, an oligonucleotide ligation assay can be performed. The microsatellite locus is first amplified by PCR. Then, different oligonucleotides can be submitted to ligation at the center of the repeat with a set of oligonucleotides covering all the possible lengths of the marker at a given locus (Zirvi et al., 1999). Another example of design of an oligonucleotide assay comprises the ligation of three oligonucleotides; a 5′ oligonucleotide hybridizing to the 5′ flanking sequence, a repeat oligonucleotide of the length of the shortest allele of the marker hybridizing to the repeated region and a set of 3′ oligonucleotides covering all the existing alleles hybridizing to the 3′ flanking sequence and a portion of the repeated region for all the alleles longer than the shortest one. For the shortest allele, the 3′ oligonucleotide exclusively hybridizes to the 3′ flanking sequence (U.S. Pat. No. 6,479,244).

The methods described herein may be performed, for example, by utilizing pre-packaged diagnostic kits comprising at least one probe nucleic acid selected from the SEQ ID of Tables 5-37, or antibody reagent described herein, which may be conveniently used, for example, in a clinical setting to diagnose patient exhibiting symptoms or a family history of a disorder or disorder involving abnormal activity of genes from Tables 2-4.

### Method to Treat an Animal Suspected of Having ADHD

The present invention provides methods of treating a disease associated with ADHD disease by expressing in vivo the nucleic acids of at least one gene from Tables 2-4. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a gene from Tables 2-4, under the control of a promoter, then express the encoded protein, thereby mitigating the effects of absent, partial inactivation, or abnormal expression of a gene from Tables 2-4.

Such gene therapy procedures have been used to correct acquired and inherited genetic defects, cancer, and viral infection in a number of contexts. The ability to express artificial genes in humans facilitates the prevention and/or cure of many important human disorders, including many disorders which are not amenable to treatment by other therapies (for a review of gene therapy procedures, see Anderson, 1992; Nabel & Feigner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994).

Delivery of the gene or genetic material into the cell is the first critical step in gene therapy treatment of a disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987; U.S. Pat. No. 4,797,368; WO 93/24641; Kotin, 1994; Muzyczka, 1994). Construction of recombinant AAV vectors is described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem et al., 1997; and Dranoff et al., 1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system (Wagner et al., 1998, Kearns et al., 1996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply the deleted gene function in trans. Ad vectors can transduce multiple types of tissues in vivo, including nondividing, differentiated cells such as those found in the liver, kidney and muscle tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cells expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids from Tables 2-4 are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski et al., 1989). The present invention is not limited to any method of administering such nucleic acids, but preferentially uses the methods described herein.

The present invention further provides other methods of treating ADHD disease such as administering to an individual having ADHD disease an effective amount of an agent that regulates the expression, activity or physical state of at least one gene from Tables 2-4. An “effective amount” of an agent is an amount that modulates a level of expression or activity of a gene from Tables 2-4, in a cell in the individual at least about 10%, at least about 20%, at least about 30%, at least about 40%, at least about 50%, at least about 60%, at least about 70%, at least about 80% or more, compared to a level of the respective gene from Tables 2-4 in a cell in the individual in the absence of the compound. The preventive or therapeutic agents of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen. The therapeutic efficacy of the treatment may be monitored by observing various parts of the brain and or body, or any other monitoring methods known in the art. Other ways of monitoring efficacy can be, but are not limited to monitoring inattention and/or hyperactive symptoms, or any other ADHD symptom described herein.

The present invention further provides a method of treating an individual clinically diagnosed with ADHDs' disease. The methods generally comprises analyzing a biological sample that includes a cell, in some cases, a brain cell, from an individual clinically diagnosed with ADHD disease for the presence of modified levels of expression of at least 1 gene, at least 10 genes, at least 50 genes, at least 100 genes, or at least 200 genes from Tables 2-4. A treatment plan that is most effective for individuals clinically diagnosed as having a condition associated with ADHD disease is then selected on the basis of the detected expression of such genes in a cell. Treatment may include administering a composition that includes an agent that modulates the expression or activity of a protein from Tables 2-4 in the cell. Information obtained as described in the methods above can also be used to predict the response of the individual to a particular agent. Thus, the invention further provides a method for predicting a patient's likelihood to respond to a drug treatment for a condition associated with ADHD disease, comprising determining whether modified levels of a gene from Tables 2-4 is present in a cell, wherein the presence of protein is predictive of the patient's likelihood to respond to a drug treatment for the condition. Examples of the prevention or improvement of symptoms accompanied by ADHD disease that can monitored for effectiveness include prevention or improvement of inattention and/or hyperactivity, or any other ADHD related symptom described herein.

The invention also provides a method of predicting a response to therapy in a subject having ADHD disease by determining the presence or absence in the subject of one or more markers associated with ADHD disease described in Tables 5-37, diagnosing the subject in which the one or more markers are present as having ADHD disease, and predicting a response to a therapy based on the diagnosis e.g., response to therapy may include an efficacious response and/or one or more adverse events. The invention also provides a method of optimizing therapy in a subject having ADHD disease by determining the presence or absence in the subject of one or more markers associated with a clinical subtype of ADHD disease, diagnosing the subject in which the one or more markers are present as having a particular clinical subtype of ADHD disease, and treating the subject having a particular clinical subtype of ADHD disease based on the diagnosis. As an example, treatment for the inattentive subtype of ADHD.

Thus, while there are a number of treatments for ADHD disease currently available, they all are accompanied by various side effects, high costs, and long complicated treatment protocols, which are often not available and effective in a large number of individuals. Accordingly, there remains a need in the art for more effective and otherwise improved methods for treating and preventing ADHD. Thus, there is a continuing need in the medical arts for genetic markers of ADHD disease and guidance for the use of such markers. The present invention fulfills this need and provides further related advantages.

### EXAMPLES

### Example 1

### Identification of Cases and Controls

All individuals were sampled from the Quebec founder population (QFP). Membership in the founder population was defined as having four grandparents of the affected child having French Canadian family names and being born in the Province of Quebec, Canada or in adjacent areas of the Provinces of New Brunswick and Ontario or in New England or New York State. The Quebec founder population is expected to have two distinct advantages over general populations for LD mapping: 1) increased LD resulting from a limited number of generations since the founding of the population and 2) increased genetic alleic homogeneity because of the restricted number of founders (estited 2600 effective founders, Charbonneau et al. 1987). Reduced allelic heterogeneity will act to increase relative risk imparted by the remaining alleles and so increase the power of case/control studies to detect genes and gene alleles involved in complex disorders within the Quebec population. The specific combination of age in generations, optimal number of founders and large present population size makes the QFP optimal for LD-based gene mapping.

All enrolled QFP subjects (patients and controls) provided a 20 ml blood sample (2 barcoded tubes of 10 ml). Samples were processed immediately upon arrival at the laboratory. All samples were scanned and logged into a LabVantage Laboratory Information Management System (LIMS), which served as a hub between the clinical data management system and the genetic analysis system. Following centrifugation, the buffy coat containing the white blood cells was isolated from each tube. Genomic DNA was extracted from the buffy coat from one of the tubes, and stored at 4° C. until required for genotyping. DNA extraction was performed with a commercial kit using a guanidine hydrochloride based method (FlexiGene, Qiagen) according to the manufacturer's instructions. The extraction method yielded high molecular weight DNA, and the quality of every DNA sample was verified by agarose gel electrophoresis. Genomic DNA appeared on the gel as a large band of very high molecular weight. The remaining two buffy coats were stored at ±80° C. as backups.

The QFP samples were collected as family trios consisting of ADHD disease subjects and two first degree relatives. 459 Parent, Parent, Child (PPC) trios were used for the analysis reported here. For the 459 trios used in the genome wide scan, these included 93 daughters and 376 sons. The child is always the affected member of the trio, so, the two non-transmitted parental chromosomes (one from each parent) were used as controls. The recruitment of trios allowed a more precise determination of long extended haplotypes.

### Example 2

### Genome Wide Association

Genotyping was performed using the QLDM-Max SNP map using IIlumina's Infinium-II technology Single Sample Beadchips. The QLDM-Max map contains 374,187 SNPs. The SNPs are contained in the Illumina HumanHap-300 arrays plus two custom SNP sets of approximately 30,000 markers each. The HumanHap-300 chip includes 317,503 tag SNPs derived from the Phase I HapMap data. The additional (approx.) 60,000 SNPs were selected by to optimize the density of the marker map across the genome matching the LD pattern in the Quebec Founder Population, as established from previous studies at Genizon, and to fill gaps in the Illumina HumanHap-300 map. The SNPs were genotyped on the 459 trios for a total of ˜515,255,499 genotypes.

The genotyping information was entered into a Unified Genotype Database (a proprietary database under development) from which it was accessed using custom-built programs for export to the genetic analysis pipeline. Analyses of these genotypes were performed with the statistical tools described in Example 3. The GWS and the different analyses permitted the identification of 288 candidate chromosomal regions linked to ADHD disease (Table 1).

### Example 3

### Genetic Analysis

1. Dataset Quality Assessment

Prior to performing any analysis, the dataset from the GWS was verified for completeness of the trios. The programs FamCheck and FamPull removed any trios with abnormal family structure or missing individuals (e.g. trios without a proband, duos, singletons, etc.), and calculated the total number of complete trios in the dataset. The trios were also tested to make sure that no subjects within the cohort were related more closely than second cousins (6 meiotic steps).

Subsequently, the program DataCheck2.1 was used to calculate the following statistics per marker and per family:

Minor allele frequency (MAF) for each marker; Missing values for each marker and family; Hardy Weinberg Equilibrium for each marker; and Mendelian segregation error rate.

The following acceptance criteria were applied for internal analysis purposes:

MAF>4%;

Missing values <1%;

Observed non-Mendelian segregation<0.33%;

Non significant deviation in allele frequencies from Hardy Weinberg equilibrium.

Markers or families not meeting these criteria were removed from the dataset in the following step. Analyses of variance were performed using the algorithm GenAnova, to assess whether families or markers have a greater effect on missing values and/or non-Mendelian segregation. This was used to determine the smallest number of data points to remove from the dataset in order to meet the requirements for missing values and non-Mendelian segregation. The families and/or markers were removed from the dataset using the program DataPull, which generates an output file that is used for subsequent analysis of the genotype data.

2. Phase Determination

The program PhaseFinderSNP2.0 was used to determine phase from trio data on a marker-by-marker, trio-by-trio basis. The output file contains haplotype data for all trio members, with ambiguities present when all trio members are heterozygous or where data is missing. The program AllHaps2PatCtrl was then used to determine case and control haplotypes and to prepare the data in the proper input format for the next stage of analysis, using the expectation maximization algorithm, PL-EM, to call phase on the remaining ambiguities. This stage consists of several modules for resolution of the remaining phase ambiguities. PLEMPre was first used to recode the haplotypes for input into the PL-EM algorithm in 11-marker blocks. The haplotype information was encoded as genotypes, allowing for the entry of known phase into the algorithm; this method limits the possible number of estimated haplotypes conditioned on already known phase assignments. The PL-EM algorithm was used to estimate haplotypes from the “pseudo-genotype” data in 11-marker windows, advancing in increments of one marker across the chromosome. The results were then converted into multiple haplotype files using the program PLEMPost. Subsequently PLEMBlockGroup was used to convert the individual 11-marker block files into one continuous block of haplotypes for the entire chromosome, and to generate files for further analysis by LDSTATS and SINGLETYPE. PLEMMerge takes the consensus estimation of the allele call at each marker over all separate estimations (most markers are estimated 11 different times as the 11 marker blocks pass over their position).

3. Haplotype Association Analysis

Haplotype association analysis was performed using the program LDSTATS. LDSTATS tests for association of haplotypes with the disease phenotype. The algorithms LDSTATS (v2.0) and LDSTATS (v4.0) define haplotypes using multi-marker windows that advance across the marker Map in one-marker increments. Windows can contain any odd number of markers specified as a parameter of the algorithm. Other marker windows can also be used. At each position the frequency of haplotypes in cases and controls was calculated and a chi-square statistic was calculated from case control frequency tables. For LDSTATS v2.0, the significance of the chi-square for single marker and 3-marker windows was calculated as Pearson's chi-square with degrees of freedom. Larger windows of multi-allelic haplotype association were tested using Smith's normalization of the square root of Pearson's Chi-square. In addition, LDSTATS v2.0 calculates Chi-square values for the transmission disequilibrium test (TDT) for single markers in situations where the trios consisted of parents and an affected child.

LDSTATS v4.0 calculates significance of chi-square values using a permutation test in which case-control status is randomly permuted until 350 permuted chi-square values are observed that are greater than or equal to chi-square value of the actual data. The P value is then calculated as 350/the number of permutations required.

Table 5.1 lists the results for association analysis using LDSTATs (v2.0 and v4.0) for the candidate regions described above based on the genome wide scan genotype data for 459 QFP trios. For each one of these regions, we report in Table 5.2 the allele frequencies and the relative risk (RR) for the haplotypes contributing to the best signal at each SNP in the region. The best signal at a given location was determined by comparing the significance (p-value) of the association with ADHD disease for window sizes of 1, 3, 5, 7, and 9 SNPs, and selecting the most significant window. For a given window size at a given location, the association with ADHD disease was evaluated by comparing the overall distribution of haplotypes in the cases with the overall distribution of haplotypes in the controls. Haplotypes with a relative risk greater than one increase the risk of developing ADHD disease while haplotypes with a relative risk less than one are protective and decrease the risk.

4. Singletype Analysis

The SINGLETYPE algorithm assesses the significance of case-control association for single markers using the genotype data from the laboratory as input in contrast to LDSTATS single marker window analyses, in which case-control alleles for single markers from estimated haplotypes in file, hapatctr.txt, as input. SINGLETYPE calculates P values for association for both alleles, 1 and 2, as well as for genotypes, 11, 12, and 22, and plots these as −log₁₀ P values for significance of association against marker position. Significance of dominance/recessive models is also assessed for each marker.

5. Conditional Haplotype Analyses

Conditional haplotype analyses were performed on subsets of the original set of 459 cases using the program LDSTATS (v2.0). The selection of a subset of cases and their matched controls was based on the carrier status of cases at a gene or locus of interest. We selected the locus LOC643182 on chromosome 3 and genes KCNAB1 on chromosome 3, ODZ3 on chromosome 4, ODZ2 on chromosome 5, GRID1 on chromosome 10, TAF4 on chromosome 20 and SLC6A14 on chromosome X, based on our association findings using LDSTATS (v2.0). The most significant association signal in LOC643182, using build 36, was obtained with a haplotype window of size 5 containing SNPs corresponding to SEQ IDs 14447, 14448, 14449, 14450, 14451 (see Table below for conversion to the specific DNA alleles used). A reduced haplotype diversity was observed and we selected a set of risk and a set of protective haplotypes for conditional analyses. The risk set consisted of haplotypes 12222, 11221, and 21212 but not the haplo-genotypes 11221/11122 and 21212/11122. Using this set, we partitioned the cases into two groups; the first group consisting of those cases that were carrier of a risk haplo-genotype and the second group consisting of the remaining cases, the non-carriers. The resulting sample sizes were respectively 222 and 230. LDSTATS (v2.0) was run in each group and regions showing association with ADHD are reported in Table 37.1. Regions associated with ADHD in the group of carriers (Has LOC643182-1_cr) indicate the presence of an epistatic interaction between risk factors in those regions and risk factors in LOC643182 (Table 37.2). The protective set consisted of haplotype 11122 but not the haplo-genotypes 11122/12222 and 11122/11221. Using this set, we partitioned the cases into two groups; the first group consisting of those cases that were carrier of a protective haplo-genotype and the second group consisting of the remaining cases, the non-carriers. The resulting sample sizes were respectively 126 and 326. LDSTATS (v2.0) was run in each group and regions showing association with ADHD are reported in Table 10.1. Regions associated with ADHD in the group of non-carriers (Not LOC643182-1_cp) indicate the presence of an epistatic interaction between risk factors in those regions and risk factors in LOC643182 (Table 10.2).

A second conditional analysis was performed using gene KCNAB1 on chromosome 3. The most significant association, using build 36, was obtained with a haplotype window of size 5 containing SNPs corresponding to SEQ IDs 15002, 15003, 15004, 15005, 15006 (see Table below for conversion to the specific DNA alleles used). A reduced haplotype diversity was observed and we selected a set of protective haplotypes for conditional analyses. The set consisted of haplo-genotypes 11121/21212, 11121/22222, 11121/11121 and 11121/22212. Using the risk set, we partitioned the cases into two groups; the first group consisting of those cases that were carrier of a protective haplo-genotype and the second group consisting of the remaining cases, the non-carriers. The resulting sample sizes were respectively 55 and 397. LDSTATS (v2.0) was run in each group and regions showing association with ADHD are reported in Table 11.1. Regions associated with ADHD in the group of non-carriers (Not LOC643182-2_cp) indicate the presence of an epistatic interaction between risk factors in those regions and risk factors in KCNAB1 (Table 11.2).

A third conditional analysis was performed using gene ODZ3 on chromosome 4. The most significant association in ODZ3, using build 36, in the subset of cases without the Combined sub-phenotype, was obtained with a haplotype window of size 5 containing SNPs corresponding to SEQ 15723, 15724, 15725, 15726, 15727 (see Table below for conversion to the specific DNA alleles used). A reduced haplotype diversity was observed and we selected a set of risk and a set of protective haplo-genotypes for conditional analyses. The risk set consisted of haplotypes 12122, 21221, 22221, 22112 but not haplo-genotype 22221/22122. The protective set consisted of haplotypes 22122, 12121, 21121 but not haplo-genotypes 22122/12122, 22122/21221, 22122/22112, 21121/22221 and 21121/22112. Using the risk set, we partitioned the cases into two groups; the first group consisting of those cases that were carrier of a risk haplo-genotype and the second group consisting of the remaining cases, the non-carriers. The resulting sample sizes were respectively 91 and 107. LDSTATS (v2.0) was run in each group and regions showing association with ADHD are reported in Tables 21.2 and 25.2. Regions associated with ADHD in the group of carriers (Has ODZ3-1_cr) indicate the presence of an epistatic interaction between risk factors in those regions and risk factors in ODZ3 (Table 21.3). Regions associated with ADHD in the group of non-carriers (Not ODZ3-1_cr) indicate the existence of risk factors acting independently of ODZ3 (Table ODZ3.3). Using the protective set, we partitioned the cases into two groups; the first group consisting of those cases that were carrier of a protective haplo-genotype and the second group consisting of the remaining cases, the non-carriers. The resulting sample sizes were respectively 72 and 126. LDSTATS (v2.0) was run in each group and regions showing association with ADHD are reported in Table 20.2. Regions associated with ADHD in the group of carriers (Has ODZ3-1_cp) indicate the presence of an epistatic interaction between risk factors in those regions and risk factors in ODZ3 (Table 20.3).

A fourth conditional analysis was performed using gene ODZ2 on chromosome 5. The most significant association in ODZ2, using build 36, in the subset of cases without the Mainly Inattentive sub-phenotype, was obtained with a haplotype window of size 7 containing SNPs corresponding to SEQ IDs 16305, 16306, 16307, 16308, 16309, 16310, 16311 (see Table below for conversion to the specific DNA alleles used). A reduced haplotype diversity was observed and we selected a set of risk and a set of protective haplo-genotypes for conditional analyses. The risk set consisted of haplotypes 1122212, 1122112, 2211122, 2122112, 1111112, 1111122, 1222122 and haplo-genotype 1222121/1222121 but not haplo-genotypes 1122212/1211122, 2211122/1211122 and 2122112/1222121. The protective set consisted of haplo-genotypes 1211122/1211122, 1211122/2211122, 1211122/1222121, 2122112/1222121. Using the risk set, we partitioned the cases into two groups; the first group consisting of those cases that were carrier of a risk haplo-genotype and the second group consisting of the remaining cases, the non-carriers. The resulting sample sizes were respectively 167 and 130. LDSTATS (v2.0) was run in each group and regions showing association with ADHD are reported in Table 28.2. Regions associated with ADHD in the group of non-carriers (Not ODZ3-1_cr) indicate the existence of risk factors acting independently of ODZ2 (Table 28.3). Using the protective set, we partitioned the cases into two groups; the first group consisting of those cases that were carrier of a protective haplo-genotype and the second group consisting of the remaining cases, the non-carriers. The resulting sample sizes were respectively 110 and 187. LDSTATS (v2.0) was run in each group and regions showing association with ADHD are reported in Tables 22.2 and 26.2. Regions associated with ADHD in the group of carriers (Has ODZ3-1_cp) indicate the existence of risk factors acting independently of ODZ2 (Table 22.3). Regions associated with ADHD in the group of non-carriers (Not ODZ3-1_cp) indicate the presence of an epistatic interaction between risk factors in those regions and risk factors in ODZ2 (Table 26.3).

A fifth conditional analysis was performed using gene ODZ2 on chromosome 5. The most significant association in ODZ2, using build 36, in the subset of cases with the Combined sub-phenotype, was obtained with a haplotype window of size 7 containing SNPs corresponding to SEQ IDs 16321, 16322, 16323, 16324, 16325, 16326, 16327 (see Table below for conversion to the specific DNA alleles used). A reduced haplotype diversity was observed and we selected a set of risk and a set of protective haplo-genotypes for conditional analyses. The risk set consisted of haplotypes 2122112, 1221222, 1211122, 2111122 and haplo-genotypes 1211111/1211111 and 2121111/2121111 but not haplo-genotypes 2122112/1222112, 1221222/1222112, 1221222/1221111, 1211122/1221111, 1211122/2111111, 2111122/1221111. The protective set consisted of haplo-genotypes 1222112/1222112, 1222112/2221111, 1222112/1221222, 1222112/1221111, 1222112/1212111, 1222112/2121111, 1222112/1221112, 1222112/1211111, 2221111/2221111, 2221111/1221111, 2221111/2121111, 2221111/2111111, 2221111/1211111, 1221111/1221111, 1221111/1212111, 1221111/2121111, 1221111/2111111, 1221111/1222111, 1221111/1221112, 1221111/1222222, 1221111/1211111, 1221111/2122222, 1221111/1221211, 1221111/2211122 and 1222111/1211111. Using the risk set, we partitioned the cases into two groups; the first group consisting of those cases that were carrier of a risk haplo-genotype and the second group consisting of the remaining cases, the non-carriers. The resulting sample sizes were respectively 100 and 161. LDSTATS (v2.0) was run in each group and regions showing association with ADHD are reported in Tables 24.2 and 30.2. Regions associated with ADHD in the group of non-carriers (Has ODZ3-2_cr) indicate the presence of an epistatic interaction between risk factors in those regions and risk factors in ODZ2 (Table 24.3). Regions associated with ADHD in the group of carriers (Not ODZ3-2_cr) indicate the existence of risk factors acting independently of ODZ2 (Table 30.3). Using the protective set, we partitioned the cases into two groups; the first group consisting of those cases that were carrier of a protective haplo-genotype and the second group consisting of the remaining cases, the non-carriers. The resulting sample sizes were respectively 77 and 184. LDSTATS (v2.0) was run in each group and regions showing association with ADHD are reported in Tables 23.2 abd 29.1. Regions associated with ADHD in the group of carriers (Has ODZ3-2_cp) indicate the existence of risk factors acting independently of ODZ2 (Table 23.3). Regions associated with ADHD in the group of non-carriers (Not ODZ3-2_cp) indicate the presence of an epistatic interaction between risk factors in those regions and risk factors in ODZ2 (Table 29.2).

A sixth conditional analysis was performed using gene GRID1 on chromosome 10. The most significant association in GRID1, using build 36, was obtained with a haplotype window of size 9 containing SNPs corresponding to SEQ IDs 19043, 19044, 19045, 19046, 19047, 19048, 19049, 19050, 19051 (see Table below for conversion to the specific DNA alleles used). A reduced haplotype diversity was observed and we selected a set of risk and a set of protective haplo-genotypes for conditional analyses. The risk set consisted of haplo-genotypes 112111111/212111111, 211222222/212111111, 212111111/212222212, 112111111/112111111, 112211122/212111111, The protective set consisted of haplo-genotypes 122111111/212111111, 212111111/212111212, 112111112/212111111, 112222212/212111111, 121222222/212111111, 122111112/212111111. Using the risk set, we partitioned the cases into two groups; the first group consisting of those cases that were carrier of a risk haplo-genotype and the second group consisting of the remaining cases, the non-carriers. The resulting sample sizes were respectively 97 and 355. LDSTATS (v2.0) was run in each group and regions showing association with ADHD are reported in Tables 6.1 and 31.1. Regions associated with ADHD in the group of carriers (Has GRID1-1_cr) indicate the presence of an epistatic interaction between risk factors in those regions and risk factors in GRID1 (Table 6.2). Regions associated with ADHD in the group of non-carriers (Not GRID1-1_cr) indicate the existence of risk factors acting independently of GRID1 (Table 31.2). Using the protective set, we partitioned the cases into two groups; the first group consisting of those cases that were carrier of a protective haplo-genotype and the second group consisting of the remaining cases, the non-carriers. The resulting sample sizes were respectively 34 and 418. LDSTATS (v2.0) was run in each group and regions showing association with ADHD are reported in Table 12.1. Regions associated with ADHD in the group of non-carriers (Not GRID1-1_cp) indicate the presence of an epistatic interaction between risk factors in those regions and risk factors in GRID1 (Table 12.2).

A seventh conditional analysis was performed using gene TAF4 on chromosome 20. The most significant association in TAF4, using build 36, was obtained with a haplotype window of size 3 containing SNPs corresponding to SEQ ID 22583, 22584, 22585 (see Table below for conversion to the specific DNA alleles used). A reduced haplotype diversity was observed and we selected a set of risk and a set of protective haplotypes for conditional analyses. The risk set consisted of haplotype 122 and haplo-genotypes 111/222, 212/222, 111/111 and 111/112. The protective set consisted of haplotype 211 but excluding haplo-genotypes 211/122, 211/221 and 211/111 due to dominance effects. Using the risk set, we partitioned the cases into two groups; the first group consisting of those cases that were carrier of a risk haplo-genotype and the second group consisting of the remaining cases, the non-carriers. The resulting sample sizes were respectively 135 and 317. LDSTATS (v2.0) was run in each group and regions showing association with ADHD are reported in Tables 7.1 and 14.1. Regions associated with ADHD in the group of carriers (Has TAF4-1_cr) indicate the presence of an epistatic interaction between risk factors in those regions and risk factors in TAF4 (Table 7.2). Regions associated with ADHD in the group of non-carriers (Not C20-1_cr) indicate the existence of risk factors acting independently of TAF4 (Table 14.2). Using the protective set, we partitioned the cases into two groups; the first group consisting of those cases that were carrier of a protective haplo-genotype and the second group consisting of the remaining cases, the non-carriers. The resulting sample sizes were respectively 115 and 337. LDSTATS (v2.0) was run in each group and regions showing association with ADHD are reported in Table 13.1. Regions associated with ADHD in the group of non-carriers (Not TAF4-1_cp) indicate the presence of an epistatic interaction between risk factors in those regions and risk factors in TAF4 (Table 13.2).

An eighth conditional analysis was performed using gene SLC6A14 on chromosome X. The most significant association signal in SLC6A14, using build 36, was obtained with a haplotype window of size 5 containing SNPs corresponding to SEQ IDs 23307, 23308, 23309, 23310, 23311 (see Table below for conversion to the specific DNA alleles used). A reduced haplotype diversity was observed and we selected a set of risk and two sets of protective haplotypes for conditional analyses. The risk set consisted of haplotypes 21211 and 21121. The protective set consisted of haplotypes 12122 and 12121. Using the risk set, we partitioned the cases into two groups; the first group consisting of those cases that were carrier of a risk haplo-genotype and the second group consisting of the remaining cases, the non-carriers. The resulting sample sizes were respectively 66 and 389. LDSTATS (v2.0) was run in each group and regions showing association, with ADHD are reported in Table 17.1. Regions associated with ADHD in the group of non-carriers (Not SLC6A14-1_cr2) indicate the existence of risk factors acting independently of SLC6A14 (Table 17.2). Using the protective set, we partitioned the cases into two groups; the first group consisting of those cases that were carrier of a protective haplotype and the second group consisting of the remaining cases, the non-carriers. The resulting sample sizes were respectively 168 and 287. LDSTATS (v2.0) was run in each group and regions showing association with ADHD are reported in Tables 8.1 and 15.1. Regions associated with ADHD in the group of non-carriers (Not SLC6A14-1_cp2) indicate the presence of an epistatic interaction between risk factors in those regions and risk factors in SLC6A14 (Table 15.2). Regions associated with ADHD in the group of carriers (Has SLC6A14-1_cp2) indicate the existence of risk factors acting independently of SLC6A14 (Table 8.2). In addition, we considered a set of risk and a set of protective haplotypes in gene SLC6A14, based on the association results using LDSTATS (v04). The most significant association signal in SLC6A14, using build 36, was obtained with a single SNP corresponding to SEQ ID 11406 (see Table below for conversion to the specific DNA alleles used). Allele 1 was the risk allele, however because of dominance effect in heterozygote female we also considered the protective allele 2 to partition the cases. Using the risk allele, we partitioned the cases into two groups; the first group consisting of those cases that were carrier of allele 1 and the second group consisting of the remaining cases, the females 2/2 and male 2, the non-carriers. The resulting sample sizes were respectively 87 and 368. Using the protective allele 2, the resulting sample sizes were respectively 395 and 60. LDSTATS (v2.0) was run in each group and regions showing association with ADHD are reported in Table 9.1, 18.1 and 19.1. Regions associated with ADHD in the group of non-carriers of allele 1 (Not SLC6A14-1a_cr1 and Not SLC6A14-1a_cp1) indicate the presence of an epistatic interaction between risk factors in those regions and risk factors in SLC6A14 (Tables 19.2 and 18.2). Regions associated with ADHD in the group of carriers of allele 1 (has SLC6A14-1a_cr1) indicate the existence of risk factors acting independently of SLC6A14 (Table 9.2).

For each region that was associated with ADHD in the conditional analyses, we report in the allele frequencies and the relative risk (RR) for the haplotypes contributing to the best signal at each SNP in the region. The best signal at a given location was determined by comparing the significance (p-value) of the association with ADHD for window sizes of 1, 3, 5, 7, and 9 SNPs, and selecting the most significant window. For regions showing association to single SNPs we report on window of size 1 only. For a given window size at a given location, the association with ADHD was evaluated by comparing the overall distribution of haplotypes in the cases with the overall distribution of haplotypes in the controls. Haplotypes with a relative risk greater than one increase the risk of developing ADHD while haplotypes with a relative risk less than one are protective and decrease the risk.

DNA alleles used in haplotypes (LOC643182)

| SeqID | 14447 | 14448 | 14449 | 14450 | 14451 |
|---|---|---|---|---|---|
| Position | 5097629 | 5101013 | 5101391 | 5104769 | 5107540 |
| Alleles | T/C | A/G | T/G | A/G | T/C |
| 12222 | T | G | G | G | C |
| 11221 | T | A | G | G | T |
| 21212 | C | A | G | A | C |
| 11122 | T | A | T | G | C |

DNA alleles used in haplotyes KCNAB1

| SeqID | 15002 | 15003 | 15004 | 15005 | 15006 |
|---|---|---|---|---|---|
| Position | 157384557 | 157448444 | 157466631 | 157475203 | 157487648 |
| Alleles | C/T | A/G | A/C | C/T | C/T |
| 11121 | T | A | A | C | T |
| 21212 | C | A | C | T | C |
| 22222 | C | G | C | C | C |
| 22212 | C | G | C | T | C |

DNA alleles used in haplotypes (GRID1)

| SeqID | 19043 | 19044 | 19045 | 19046 | 19047 | 19048 | 19049 | 19050 | 19051 |
|---|---|---|---|---|---|---|---|---|---|
| Position | 87981204 | 87981896 | 87983053 | 87986431 | 87998880 | 88002203 | 88004329 | 88019566 | 88030744 |
| Alleles | G/A | C/A | G/A | A/G | T/C | A/C | C/T | T/C | C/T |
| 112111111 | A | A | G | A | T | A | T | T | T |
| 112111112 | A | A | G | A | T | A | T | T | C |
| 112211122 | A | A | G | G | T | A | T | C | C |
| 112222212 | A | A | G | G | C | C | C | T | C |
| 121222222 | A | C | A | G | C | C | C | C | C |
| 122111111 | A | C | G | A | T | A | T | T | T |
| 122111112 | A | C | G | A | T | A | T | T | C |
| 211222222 | G | A | A | G | C | C | C | C | C |
| 212111111 | G | A | G | A | T | A | T | T | T |
| 212111212 | G | A | G | A | T | A | C | T | C |
| 212222212 | G | A | G | 6 | C | C | C | T | C |

DNA alleles used in haplotypes (TAF4)

| SeqID | 22583 | 22584 | 22585 |
|---|---|---|---|
| Position | 60083924 | 60091799 | 60095481 |
| Alleles | C/T | A/G | A/G |
| 211 | C | A | A |
| 122 | T | G | G |
| 221 | C | G | A |
| 111 | T | A | A |
| 222 | C | G | G |
| 212 | C | A | G |
| 112 | T | A | G |

DNA alleles used in haplotypes (SLC6A14)

| SeqID | 23307 | 23308 | 23309 | 23310 | 23311 |
|---|---|---|---|---|---|
| Position | 115464677 | 115465239 | 115479909 | 115480867 | 115485218 |
| Alleles | A/C | A/G | A/C | G/A | C/T |
| 12122 | A | G | A | G | C |
| 12121 | A | G | A | G | T |

| SeqId | 11406 |
|---|---|
| Position | 115465239 |
| Alleles | A/G |
| RISK ALLELE | A |
| 1 | |
| PROTECTIVE | G |
| ALLELE 2 | |

DNA alleles used in haplotypes (ODZ3)

| SeqID | 15723 | 15724 | 15725 | 15726 | 15727 |
|---|---|---|---|---|---|
| Position | 183922396 | 183923229 | 183926660 | 183928473 | 183928541 |
| Alleles | A/G | A/C | A/G | T/C | A/G |
| 22122 | G | C | A | C | G |
| 12121 | A | C | A | C | A |
| 21121 | G | A | A | C | A |
| 12122 | A | C | A | C | G |
| 21221 | G | A | G | C | A |
| 22221 | G | C | G | C | A |
| 22112 | G | C | A | T | G |

DNA alleles used in haplotypes (ODZ2)

| SeqID | 16305 | 16306 | 16307 | 16308 | 16309 | 16310 | 16311 |
|---|---|---|---|---|---|---|---|
| Position | 166726668 | 166730514 | 166741180 | 166741993 | 166753729 | 166756680 | 166770180 |
| Alleles | A/G | A/G | T/C | A/C | T/G | T/G | T/G |
| 1211122 | A | G | T | A | T | G | G |
| 2211122 | G | G | T | A | T | G | G |
| 2122112 | G | A | C | C | T | T | G |
| 1222121 | A | G | C | C | T | G | T |
| 1122212 | A | A | C | C | G | T | G |
| 1122112 | A | A | C | C | T | T | G |
| 1111112 | A | A | T | A | T | T | G |
| 1111122 | A | A | T | A | T | G | G |
| 1222122 | A | G | C | C | T | G | G |
| SeqID | 16321 | 16322 | 16323 | 16324 | 16325 | 16326 | 16327 |
| Position | 166975676 | 166988514 | 166992037 | 166992322 | 166996825 | 167002992 | 167012099 |
| Alleles | A/G | A/G | T/C | T/C | A/C | T/C | T/C |
| 1222112 | A | G | C | C | A | T | C |
| 2221111 | G | G | C | T | A | T | T |
| 1221222 | A | G | C | T | C | C | C |
| 1221111 | A | G | C | T | A | T | T |
| 1212111 | A | G | T | C | A | T | T |
| 2121111 | G | A | C | T | A | T | T |
| 1221112 | A | G | C | T | A | T | C |
| 1211111 | A | G | T | T | A | T | T |
| 2111111 | G | A | T | T | A | T | T |
| 1222111 | A | G | C | C | A | T | T |
| 1222222 | A | G | C | C | C | C | C |
| 2122222 | G | A | C | C | C | C | C |
| 1221211 | A | G | C | T | C | T | T |
| 2211122 | G | G | T | T | A | C | C |
| 2122112 | G | A | C | C | A | T | C |
| 1211122 | A | G | T | T | A | C | C |
| 2111122 | G | A | T | T | A | C | C |

6. Gender Specific Analyses

The total sample of 459 trios was subdivided into those with male affected children (368 trios) and those with female affected children (91 trios) and analyzed separately. A complete genome wide analysis was redone on each separate sample and genome wide significance was recalculated for each.

7. Sub-Phenotype Analysis

Trios with affected children who were characterized by the mainly inattentive subphenotype of ADHD (162 trios) as determined by the computerized version of the Diagnostic Interview Schedule for Children (DISC-4) according to DSM-IV criteria were analyzed separately in a second genome wide scan and genome wide significance for this scan was determined separately as well.

Trios with affected children were diagnosis as determined by the computerized version of the Diagnostic Interview Schedule for Children (DISC-4) according to DSM-IV criteria were analyzed separately in a second genome wide scan and genome wide significance for this scan was determined separately as well. It can be subdivided into three different subtypes:
Attention-deficit/hyperactivity disorder, predominantly inattentive type (mainly inattentive, 162 trios)
Attention-deficit/hyperactivity disorder, predominantly hyperactive-impulsive type (mainly hyperactive of ADHD, 36 trios)
Attention-deficit/hyperactivity disorder, combined type (combined, 261 trios)

### Example 5

### Gene Identification and Characterization

A series of gene characterization was performed for each candidate region described in Table 1. Any gene or EST mapping to the interval based on public map data or proprietary map data was considered as a candidate ADHD disease gene. The approach used to identify all genes located in the critical regions is described below.

### Public Gene Mining

Once regions were identified using the analyses described above, a series of public data mining efforts were undertaken, with the aim of identifying all genes located within the critical intervals as well as their respective structural elements (i.e., promoters and other regulatory elements, UTRs, exons and splice sites). The initial analysis relied on annotation information stored in public databases (e.g. NCBI, UCSC Genome Bioinformatics, Entrez Human Genome Browser, OMIM—see below for database URL information). Table 2 lists the genes that have been mapped to the candidate regions.

For some genes the available public annotation was extensive, whereas for others very little was known about a gene's function. Customized analysis was therefore performed to characterize genes that corresponded to this latter class. Importantly, the presence of rare splice variants and artifactual ESTs was carefully evaluated. Subsequent cluster analysis of novel ESTs provided an indication of additional gene content in some cases. The resulting clusters were graphically displayed against the genomic sequence, providing indications of separate clusters that may contribute to the same gene, thereby facilitating development of confirmatory experiments in the laboratory. While much of this information was available in the public domain, the customized analysis performed revealed additional information not immediately apparent from the public genome browsers.

A unique consensus sequence was constructed for each splice variant and a trained reviewer assessed each alignment. This assessment included examination of all putative splice junctions for consensus splice donor/acceptor sequences, putative start codons, consensus Kozak sequences and upstream in-frame stops, and the location of polyadenylation signals. In addition, conserved noncoding sequences (CNSs) that could potentially be involved in regulatory functions were included as important information for each gene. The genomic reference and exon sequences were then archived for future reference. A master assembly that included all splice variants, exons and the genomic structure was used in subsequent analyses (i.e., analysis of polymorphisms). Table 3 lists gene clusters based on the publicly available EST and cDNA clustering algorithm, ECGene.

An important component of these efforts was the ability to visualize and store the results of the data mining efforts. A customized version of the highly versatile genome browser GBrowse (http://www.gmod.org/) was implemented in order to permit the visualization of several types of information against the corresponding genomic sequence. In addition, the results of the statistical analyses were plotted against the genomic interval, thereby greatly facilitating focused analysis of gene content.

### Computational Analysis of Genes and GeneMaps

In order to assist in the prioritization of candidate genes for which minimal annotation existed, a series of computational analyses were performed that included basic BLAST searches and alignments to identify related genes. In some cases this provided an indication of potential function. In addition, protein domains and motifs were identified that further assisted in the understanding of potential function, as well as predicted cellular localization.

A comprehensive review of the public literature was also performed in order to facilitate identification of information regarding the potential role of candidate genes in the pathophysiology of ADHD disease. In addition to the standard review of the literature, public resources (Medline and other online databases) were also mined for information regarding the involvement of candidate genes in specific signaling pathways. A variety of pathway and yeast two hybrid databases were mined for information regarding protein-protein interactions. These included BIND, MINT, DIP, Interdom, and Reactome, among others. By identifying homologues of genes in the ADHD candidate regions and exploring whether interacting proteins had been identified already, knowledge regarding the GeneMaps for ADHD disease was advanced. The pathway information gained from the use of these resources was also integrated with the literature review efforts, as described above.

Genes identified in the WGAS and subsequent studies for ADHD disease (ADHD) were evaluated using the Ingenuity Pathway Analysis application (IPA, Ingenuity systems) in order to identify direct biological interactions between these genes, and also to identify molecular regulators acting on those genes (indirect interactions) that could be also involved in ADHD. The purpose of this effort was to decipher the molecules involved in contributing to ADHD. These gene interaction networks are very valuable tools in the sense that they facilitate extension of the map of gene products that could represent potential drug targets for ADHD.

### ADHD Genemap and Pathways

The GWAS and subsequent data mining analyses resulted in a compelling GeneMap that contains networks highly relevant to ADHD as well as many genes under neuronal communication. Many of the identified regions contain genes involved in biologically relevant pathways: serotonin pathway, glutamate pathway, GABA pathway, dopamine pathway, Wnt signaling, T cell signaling and neuronal potentiation. The emerging GeneMap includes signaling pathways in brain development, brain plasticity, neuronal communication, behavior, memory, anxiety and aggressiveness. Interestingly, some identified hits contain genes that tend to confirm observations that link ADHD and eyes disorders.

Neuronal communication and Synaptic transmission: Although the etiology of ADHD is currently unknown, considerable evidence implicates the catecholaminergic systems. In our GWAS, several genes are link to neurotransmission. For example, SLC6A14 is a neurotransmitter (tryptophan) transporter. Tryptophan is a precursor of serotonin which has been associated with ADHD. GRID1 (glutamate receptor) and KCNAB1 (potassium voltage-gated channel) are both involved in excitatory synaptic transmission. It is also known that KCNAB1 interacts with SNAP25, a recognized candidate gene for ADHD. TAC4 is a neurotransmitter involved in synaptic plasticity. GABRG2 is the receptor for GABA, the major inhibitory neurotransmitter in the brain. SLC6A14, GRID1, KCNAB1, TAC4 and GABRG2 (mainly inattentive subphenotype) are all genes found in our GWAS, along with CYFIP1, ARHGAP22, ODZ2 and ODZ3. CYFIP has a role in neuronal connectivity: it has been shown that CYFIP mutations affect axons and synapses leading to neuronal connectivity defects. ODZ2 and ODZ3 are both adaptor in developing and adult CNS, transported from cell body to axon, having a function in neuronal communication. ARHGAP22 is a Rho GTPase activating protein. In the CNS, Rho GTPases regulate multiple signaling pathways that influence neuronal development: Rho GTPases modulate neuronal growth cone remodeling, synaptic neurotransmitter release, dendritic spine morphogenesis, synapse formation and axonal guidance. In addition to their effects on neuronal physiology, Rho GTPases are also key regulators of neuron survival. This is biologically relevant for ADHD.

Brain development, Function and Plasticity; Neuronal plasticity requires actin cytoskeleton remodeling and local protein translation in response to extracellular signals. Mutations affecting either pathway produce neuronal connectivity defects in model organisms and mental retardation in humans.

ARHGAP22, CD247, SYNE1, MYST2, S100B (from conditional analyses), THRB and AKAP12 (both from conditional analyses), EPHA5 and FGF7 (both from mainly inattentive subphenotype) are all genes found in our GWAS that are linked to brain development and plasticity.

ARHGAP22 is a Rho GTPases and this pathway control actin reorganization (needed for neuronal plasticity). CD247 has a role in neuronal development and plasticity, and also in neuronal signaling and synaptic connectivity. SYNE1 is a scaffold protein with a potential role in neuromuscular junction and development. MYST2 is a transcriptional regulator involved in adult neurogenesis and brain plasticity. S100B, a neurotrophic factor, is also a neuron survival protein during development of the central nervous system. S100 proteins influence cellular response along the calcium-signal-transduction pathway. Several disorders are linked to altered calcium levels. S100B has been linked to several neurological diseases, including Alzheimer's disease, Down's syndrome and epilepsy. THRB is a nuclear receptor that has been associated with ADHD in linkage studies by other group and is involved in brain development and function. Thyroid hormones are important during development of the mammalian brain, acting on migration and differentiation of neuronal cells, synaptogenesis, and myelination. The thyroid hormones play a critical role in brain development, and thyroid disorders have been linked to a variety of psychiatric and neuropsychological disorders, including learning deficits, impaired attention, anxiety, and depression. EPHA5 and EPH-related receptors have been implicated in mediating development of the nervous system, and also as mediators of plasticity in the adult mammalian brain. FGF7, a growth factor, promotes presynaptic differentiation. AKAP12 is a scaffold protein involved in the localization for protein kinases during neuronal development. All of these genes are biologically relevant for ADHD.

Behavior; ADHD is a neuropsychiatric condition characterized by hyperactive-impulsive behavior and persistent inattention. Individuals with this condition experience social and academic dysfunction. In our GWAS, we found several genes related to behavior: SLC6A14, GRID1, TAC4, FZD10, CYP1B1, PRKCE (from conditional analyses), SSTR2 and NBN (both from mainly inattentive subphenotype).

Already mentioned, SLC6A14 is a neurotransmitter transporter, involved in the transport of tryptophan, the precursor of serotonin which has been associated with ADHD. Serotonin plays an important role in the regulation of mood and appetite and low levels have been associated with depression and anxiety. GRID1, a glutamate receptor, has also been reported to have a role in anxiety. The role of glutamate in anxiety disorders is becoming more recognized. Glutamate is ubiquitous within the central nervous system and has been shown to play important roles in many brain processes, including neurodevelopment (differentiation, migration and survival), learning (long term potentiation and depression), neurodegeneration (Alzheimer'.s disease) and more recently anxiety disorders. TAC4, a neurotransmitter, is expressed in areas of the brain implicated in depression, anxiety, and stress, and has a role in abnormal social behaviors in rats. PRKCE is a potential target for anxiety. FDZ10 is a receptor involved behavior and social interaction. SSTR2 is the somatostatin receptor and it has been shown that decreased concentrations of somatostatin were found in disruptive behavior disorder patients. CYP1B1 is an enzyme involved in the synthesis of steroid and it is known that sex steroid hormone gene polymorphisms and depressive symptoms are involved in women at midlife. CYP1B1 also binds estrogen receptor which is involved in psychiatric disorders. All of these genes are biologically relevant for ADHD.

### ADHD and Eye

It is important to consider that all those different genes are expressed in different tissues. Even if the majority of our genes found are expressed in the brain, maybe they are in different cell structure and are not interacting together. It is interesting to look at one specific tissue and look at the genes found in that specific tissue and their relation.

One another example to connect genes is by looking at their tissues expression and tends to link genes according to that. Beside the brain, one interesting example in ADHD is the eye. Interesting observations may link ADHD to eye related problems. It is known that there is a potential relationship between convergence insufficiency, an eye disorder that normally affects less than 5% of children, and ADHD. The symptoms of convergence insufficiency can make it hard to keep both eyes pointed and focused at a near target, making it difficult for a child to concentrate on extended reading and overlap with those of ADHD. Children with the disorder, convergence insufficiency are 3 times more likely to be diagnosed with ADHD than children without the disorder. It account for 16% incidence in ADHD population.

Interestingly, one of the genes from the GWAS is a gene involved in visual perception; IMPG1 (interphotoreceptor matrix proteoglycan 1, full cohort and male analysis). It is an eye specific structural adaptor that participates in the formation of the ordered interphotoreceptor matrix lattice that surrounds photoreceptors in the outer retinal surface. It has been shown that a mutation in the IMPG1 gene may play a causal role in benign concentric annular macular dystrophy (BCAMD). The BCAMD phenotype is initially characterized by parafoveal hypopigmentation and good visual acuity, but progresses to a retinitis pigmentosa-like phenotype.

Another gene from the GWAS is SYNE1. It is a known protein associated with an orphan disease (Cerebral ataxia) discovered in Quebec. One of the associated features is minor abnormalities in ocular saccades and pursuit.

Another gene from the GWAS coincides with a specific protein (COL4A3, male analysis) component of the basement membrane and have also been associated with an orphan disease, the Alport syndrome, which has features as muscular contractures and retinal arterial tortuosities. Up to 15% of Alport syndrome cases represent the autosomal recessive form due to mutations in either the COL4A3 or the COL4A4 gene.

Coincidentally, in a recent study aiming to investigate visual function and ocular features in children with ADHD, researchers came to the conclusion that these children's had a high frequency of opthalmologic findings, which were not significantly improved with stimulants. They presented subtle morphological changes of the optic nerve and retinal vasculature, indicating an early disturbance of the development of these structures. They found smaller optic discs and neuroretinal rim areas and decreased tortuosity of retinal arteries than that of controls. It is also important to mention here that the observed subtle morphological changes are very supportive of the presence of the IMPG1 gene in our best hits list.

Furthermore, the specific component of the basement membrane (COL4A3) has also been associated in another rare eye disease study with immunohistochemical evidence of ectopic expression of this protein in corneal endothelium. In this disease, researchers showed presence of a complex (core plus secondary) binding site for specific a transcription factor (TCF8) in the promoter of our target candidate (COL4A3). This transcription factor contains a zinc-finger homeodomain and coincidentally another protein, a zinc metalloprotease, is known to act directly on our candidate (COL4A3). The zinc metalloproteases are a diverse group of enzymes which are becoming increasingly important in a variety of biological systems. Their major function is to break down proteins. Interestingly, numerous controlled studies report cross-sectional evidence of lower zinc tissue levels (serum, red cells, hair, urine, nails) in children who have ADHD, compared to normal controls and population norms. In a recent study researchers have observed that the plasma zinc levels were significantly lower in ADHD groups than controls. Also, zinc monotherapy was significantly superior to placebo in reducing symptoms of hyperactivity, impulsivity and impaired socialization in patients with ADHD, suggesting a role of zinc deficiency in the pathogenesis of ADHD.

Moreover cardiac arrhythmia and brain MRI abnormalities were also observed in association with the defect of this specific basement membrane component (COL4A3). Another identified gene, from conditional analyses (AKAP6), a scaffold protein, is expressed in various brain regions and also in cardiac and skeletal muscle. One of the most prescribed medications to treat ADHD (amphetamine, Ritalin) has been recently reported to cause serious heart problems. Thus in the Genemap, in addition to biologically relevant pathways involved in neurotransmission a brain development and behavior, we have also identified genes that may be involved in cardiac side effects.

Other GWAS gene in the Genemap is CYP1B1, a member of the cytochrome P450 superfamily of enzymes. The cytochrome P450 proteins are monooxygenases which catalyze many reactions involved in drug metabolism and synthesis of cholesterol, steroids and other lipids. Mutations in this gene have been associated with primary congenital glaucoma; therefore it is thought that the enzyme also metabolizes a signaling molecule involved in eye development, possibly a steroid. Studies on CYP1B1 indicate its requirement for normal eye development, both in human and mouse. The distribution of the enzyme in the mouse eye is in three regions, which may reflect three different, perhaps equally important, functions in this organ. Its presence in the inner ciliary and lens epithelia appears to be necessary for normal development of the trabecular meshwork and its function in regulating intraocular pressure. Its expression in the retinal ganglion and inner nuclear layers may reflect a role in maintenance of the visual cycle. Its expression in the corneal epithelium may indicate a function in metabolism of environmental xenobiotics. Identification of CYP1B1 as the gene affected in primary congenital glaucoma was the first example in which mutations in a member of the cytochrome P450 superfamily results in a primary developmental defect. At first, it was speculated that CYP1B1 participates in the metabolism of an as-yet-unknown biologically active molecule that is a participant in eye development. Later, it has been demonstrated that a stable protein product is produced in the affected subjects, and that the mutations result in a product lacking between 189 and 254 amino acids from the C terminus. This segment harbors the invariant cysteine of all known cytochrome P450 amino sequences; in CYP1B1 it is cys470. It has been demonstrated that a cytochrome-P450-dependent arachidonate metabolite inhibits Na+, K+-ATPase in the cornea in regulating corneal transparency and aqueous humor secretion. This finding is consistent with the clouding of the cornea and increased intraocular pressure, the 2 major diagnostic criteria for primary congenital glaucoma. Also reported that mice deficient in CYP1B1 have ocular drainage structure abnormalities resembling those reported in human primary congenital glaucoma patients.

In summary, this is one example describing interesting observations using only 5 genes from our discoveries (IMPG1, SYNE1, COL4A3, AKAP6 and CYP1B1) to build potential connections aiming to support link between, ADHD, eye problems and the GWAS discoveries.

### Expression Studies

In order to determine the expression patterns for genes, relevant information was first extracted from public databases. The UniGene database, for example, contains information regarding the tissue source for ESTs and cDNAs contributing to individual clusters. This information was extracted and summarized to provide an indication in which tissues the gene was expressed. Particular emphasis was placed on annotating the tissue source for bona fide ESTs, since many ESTs mapped to Unigene clusters are artifactual. In addition, SAGE and microarray data, also curated at NCBI (Gene Expression Omnibus), provided information on expression profiles for individual genes: Particular emphasis was placed on identifying genes that were expressed in tissues known to be involved in the pathophysiology of ADHD. To complement available information about the expression pattern of candidate disease genes, a RT-PCR based semi-quantitative gene expression profiling method was used.

Total human RNA samples from 24 different tissues Total RNA sample were purchased from commercial sources (Clontech, Stratagene) and used as templates for first-strand cDNA synthesis with the High-Capacity cDNA Archive kit (Applied Biosystems) according to the manufacturer's instructions. A standard PCR protocol was used to amplify genes of interest from the original sample (50 ng cDNA); three serial dilutions of the cDNA samples corresponding to 5, 0.5 and 0.05 ng of cDNA were also tested. PCR products were separated by electrophoresis on a 96-well agarose gel containing ethidium bromide followed by UV imaging. The serial dilutions of the cDNA provided semi-quantitative determination of relative mRNA abundance. Tissue expression profiles were analyzed using standard gel imaging software (AlphaImager 2200); mRNA abundance was interpreted according to the presence of a PCR product in one or more of the cDNA sample dilutions used for amplification. For example, a PCR product present in all the cDNA dilutions (i.e. from 50 to 0.05 ng cDNA) was designated ++++ while a PCR product only detectable in the original undiluted cDNA sample (i.e., 50 ng cDNA) was designated as + or +/−, for barely detectable PCR products (see Table 38). For each target gene, one or more gene-specific primer pairs were designed to span at least one intron when possible. Multiple primer-pairs targeting the same gene allowed comparison of the tissue expression profiles and controlled for cases of poor amplification.

1.-38. (canceled)

39. A method of detecting susceptibility to ADHD disease comprising detecting at least one mutation or polymorphism in the nucleic acid molecule selected from Table 2-4 in a patient.

40. The method of claim 39, wherein said method comprises hybridizing a probe to said patient's sample of DNA or RNA under stringent conditions which allow hybridization of said probe to nucleic acid comprising said mutation or polymorphism, wherein the presence of a hybridization signal indicates the presence of said mutation or polymorphism in at least one gene from Table 2-4.

41.-48. (canceled)

49. The method of claim 39, wherein the mutation is selected from the group consisting of at least one of the SNPs from Tables 5-37, alone or in combination.

50. (canceled)

52. A method of diagnosing susceptibility to ADHD disease in an individual, comprising screening for an at-risk haplotype of at least one gene or gene region from Table 2-4, that is more frequently present in an individual susceptible to ADHD disease compared to a control individual, wherein the presence of the at-risk haplotype is indicative of a susceptibility to ADHD disease.

53. The method of claim 52 wherein the at-risk haplotype is indicative of increased risk for ADHD disease.

54. The method of claim 53, wherein the risk is increased at least about 20%.

55. The method of claim 52, wherein the at-risk haplotype is characterized by the presence of at least one single nucleotide polymorphism from Tables 5-37.

56.-65. (canceled)

66. A drug screening assay comprising: a) administering a test compound to an animal having ADHD disease, or a cell population isolated therefrom; and (b) comparing the level of gene expression of at least one gene from Table 2-4 in the presence of the test compound with the level of said gene expression in normal cells; wherein test compounds which provide the level of expression of one or more genes from Table 2-4 similar to that of the normal cells are candidates for drugs to treat ADHD disease.

67.-80. (canceled)

81. A method for predicting the efficacy of a drug for treating ADHD disease in a human patient, comprising: a) obtaining a sample of cells from the patient; b) obtaining a set of genotypes from the sample, wherein the set of genotypes comprises genotypes of one or more polymorphic loci from Tables 2-37; and c) comparing the set of genotypes of the sample with a set of genotypes associated with efficacy of the drug, wherein similarity between the set of genotypes of the sample and the set of genotypes associated with efficacy of the drug predicts the efficacy of the drug for treating ADHD disease in the patient.

82.-84. (canceled)

85. The method of claim 81, wherein the set of genotypes from the sample comprises genotypes of at least two of the polymorphic loci listed in Tables 2-37.

86. The method of claim 81 wherein the set of genotypes from the sample is obtained by hybridization to allele-specific oligonucleotides complementary to the polymorphic loci from Tables 2-37, wherein said allele-specific oligonucleotides are contained on a microarray.

87. The method of claim 86, wherein the oligonucleotides comprise nucleic acid molecules at least 95% identical to SEQ ID from Tables 2-37.

88.-117. (canceled)

118. A method for identifying a gene that regulates drug response in ADHD disease, comprising: (a) obtaining a gene expression profile for at least one gene from Table 2-4 in a resident tissue cell induced for a proinflammatory like state in the presence of the candidate drug; and (b) comparing the expression profile of said gene to a reference expression profile for said gene in a cell induced for the proinflammatory like state in the absence of the candidate drug, wherein genes whose expression relative to the reference expression profile is altered by the drug may identifies the gene as a gene that regulates drug response in ADHD disease.

119.-137. (canceled)

138. A method of assessing a patient's risk of having or developing ADHD disease, comprising (a) determining a genotype for at least one polymorphic locus from Tables 2-37 in a patient; (b) comparing said genotype of (a) to a genotype for at least one polymorphic locus from Tables 2-37 that is associated with ADHD disease; and (c) assessing the patient's risk of having or developing ADHD disease, wherein said patient has a higher risk of having or developing ADHD disease if the genotype for at least one polymorphic locus from Tables 2-37 in said patient is the same as said genotype for at least one polymorphic locus from Tables 2-37 that is associated with ADHD disease.

139.-140. (canceled)

141. The method of claim 138, wherein the at least one polymorphic locus is associated a gene listed in any one of Tables 2 to 4.

142. The method of claim 138, wherein the at least one polymorphic locus comprises a single nucleotide polymorphism listed in any one of Tables 5.1, 6.1, 7.1, 8.1, 9.1, 10.1, 11.1, 12.1, 13.1, 14.1, 15.1, 16.1, 17.1, 18.1, 19.1, 20.2, 21.2, 22.2, 23.1, 23.2, 24.2, 25.2, 26.2, 27.2, 28.2, 29.1, 29.2, 30.2, 31.1, 31.2, 32.2, 33.2, 34.2, 35.1, 35.2, 36.2, 37.1 and 37.2.

143. The method of claim 138, wherein the at least one polymorphic locus comprises an haplotype listed in any one of Tables 5.2, 6.2, 7.2, 8.2, 9.2, 10.2, 11.2, 12.2, 13.2, 14.2, 15.2, 16.2, 17.2, 18.2, 19.2, 20.3, 21.3, 22.3, 23.3, 24.3, 25.3, 26.3, 27.3, 28.3, 29.3, 30.3, 31.3, 32.3, 33.3, 34.3, 35.3, 36.3 and 37.3.

144. The method of claim 138, wherein the genotype comprises (i) a risk haplotype at locus GRID-1 and (ii) a SNP listed in Table 6.1 or an haplotype listed in Table 6.2.

145. The method of claim 138, wherein the genotype comprises (i) a risk haplotype at locus TAF4 and (ii) a SNP listed in Table 7.1 or an haplotype listed in Table 7.2.

146. The method of claim 138, wherein the genotype comprises (i) a protective haplotype at locus SLC6A14 and (ii) a SNP listed in Table 8.1 or an haplotype listed in Table 8.2.

147. The method of claim 138, wherein the genotype comprises (i) a risk haplotype at locus SLC6A14 and (ii) a SNP listed in Table 9.1 or an haplotype listed in Table 9.2.

148. The method of claim 138, wherein the genotype (i) lacks a protective haplotype at locus LOC643182 and (ii) comprises a SNP liste in Table 10.1 or 15.1 or an haplotype listed in Table 10.2. or 15.2

149. The method of claim 138, wherein the genotype (i) lacks a protective haplotype at locus KCNAB1 and (ii) comprises a SNP listed in Table 11.2 or an haplotype listed in Table 11.2.

150. The method of claim 138, wherein the genotype (i) lacks a protective haplotype at locus LOC643182 and (ii) comprises a SNP listed in Table 12.1 or an haplotype listed in Table 12.2.

151. The method of claim 138, wherein the genotype (i) lacks a protective haplotype at locus TAF4 and (ii) comprises a SNP listed in Table 13.1 or an haplotype listed in Table 13.2.

152. The method of claim 138, wherein the genotype (i) lacks a risk haplotype at locus TAF4 and (ii) comprises a SNP listed in Table 14.1 or an haplotype listed in Table 14.2.

153. The method of claim 138, wherein the patient is a female patient and the genotype comprises a SNP listed in Table 16.1 or an haplotype listed in Table 16.2. [support paragraph 414]

154. The method of claim 138, wherein the genotype (i) lacks a risk haplotype at locus SLC6A14 and (ii) comprises a SNP listed in Table 17.1 or 19.1 or an haplotype listed in Table 17.2 or 19.2.

155. The method of claim 138, wherein the genotype (i) lacks a protective haplotype at locus SLC6A14 and (ii) comprises a SNP listed in Table 18.1 or an haplotype listed in Table 18.2.

148. The method of claim 138, wherein the genotype comprises (i) a protective haplotype at locus ODZ3 and (ii) a SNP listed in Table 20.2 or 22.2 or an haplotype listed in Table 20.3 or 22.3.

147. The method of claim 138, wherein the genotype comprises (i) a risk haplotype at locus ODZ3 and (ii) a SNP listed in any one of Tables 21.2, 23.2 or 24.2 or an haplotype listed in any one of Tables 21.3, 23.3 and 24.3.

150. The method of claim 138, wherein the genotype comprises (i) a protective haplotype at locus ODZ2 and (ii) a SNP listed in Table 22.2 or an haplotype listed in Table 22.3.

151. The method of claim 138, wherein the genotype (i) lacks a risk haplotype at locus ODZ3 and (ii) comprises a SNP listed in Table 25.2 or 30.2 or an haplotype listed in Table 25.3 or 30.3.

152. The method of claim 138, wherein the genotype (i) lacks a protective haplotype at locus ODZ2 and (ii) comprises a SNP listed in Table 26.2 or an haplotype listed in Table 26.3.

153. The method of claim 138, wherein the patient is a male patient and the genotype comprises a SNP listed in Table 27.2 or a haplotype listed in Table 27.3.

154. The method of claim 138, wherein the genotype (i) lacks a risk haplotype at locus ODZ2 and (ii) comprises a SNP listed in Table 28.2 or an haplotype listed in Table 28.3.

155. The method of claim 138, wherein the genotype (i) lacks a protective haplotype at locus ODZ2 and (ii) comprises a SNP listed in Table 29.2 or an haplotype listed in Table 29.3.

156. The method of claim 138, wherein the genotype (i) lacks a risk haplotype at locus GRID-1 and (ii) comprises a SNP listed in Table 31.2 or an haplotype listed in Table 31.1.

157. The method of claim 138, wherein the patient is of the combined sub-type and the genotype comprises a SNP listed in Table 32.2 or an haplotype listed in Table 32.3.

158. The method of claim 138, wherein the patient is of the inattentive sub-type and the genotype comprises a SNP listed in Table 33.2 or an haplotype listed in Table 33.3.

159. The method of claim 138, wherein the patient is not of the combined sub-type and the genotype comprises a SNP listed in Table 34.2 or an haplotype listed in Table 34.3.

160. The method of claim 138, wherein the patient is not of the hyperactive sub-type and the genotype comprises a SNP listed in Table 35.2 or an haplotype listed in Table 35.3.

161. The method of claim 138, wherein the patient is not of the combined sub-type and the genotype comprises a SNP listed in Table 36.2 or an haplotype listed in Table 36.3.

162. The method of claim 138, wherein the genotype comprises (i) a risk haplotype at locus LOC643182 and (ii) a SNP listed in Table 37.2 or an haplotype listed in Table 31.2.
